SEQUENCE LISTING (10 sequences)

SEQ ID NO: 1 - protein, 7 aa, synthetic construct
VFEHIGK

SEQ ID NO: 2 - protein, 9 aa, synthetic construct
NVAVDELSR

SEQ ID NO: 3 - protein, 14 aa, synthetic construct
EIEIDIEPTDKVER

SEQ ID NO: 4 - protein, 13 aa, synthetic construct
TAFQEALDAAGDK

SEQ ID NO: 5 - protein, 16 aa, synthetic construct
  source 1..16: note = VP1
NTPVPADPPTTFNQSK

SEQ ID NO: 6 - protein, 7 aa, synthetic construct
  source 1..7: note = Q9H254
LELLAQR

SEQ ID NO: 7 - protein, 19 aa, synthetic construct
  source 1..19: note = VP1
NSLANPGIAMATHKDDEER

SEQ ID NO: 8 - protein, 9 aa, synthetic construct
  source 1..9: note = VP1
HPPPQILIK

SEQ ID NO: 9 - protein, 20 aa, synthetic construct
  source 1..20: note = VP1
VSTTTGQNNNSNFAWTAGTK

SEQ ID NO: 10 - protein, 10 aa, synthetic construct
  source 1..10: note = P02768
LVNEVTEFAK

(19) United States
(12) Patent Application Publication
(10) Pub. No.: US 2025/0067748 A1
(43) Pub. Date: Feb. 27, 2025

(54) HOST CELL PROTEIN ANALYSIS OF AAV USING PROTEOMINER TECHNOLOGY

(71) Applicant: Regeneron Pharmaceuticals, Inc., Tarrytown, NY (US)

(72) Inventors: Sisi Zhang, White Plains, NY (US); Hui Xiao, Ridgewood, NJ (US); Ning Li, New Canaan, CT (US)

(21) Appl. No.: 18/814,074

(22) Filed: Aug. 23, 2024

Related U.S. Application Data
(60) Provisional application No. 63/534,637, filed on Aug. 25, 2023.

(51) Int. Cl. G01N 33/68

(52) U.S. Cl. CPC G01N 33/6842; G01N 33/6851

(57) ABSTRACT

The present disclosure provides methods for enriching, identifying, and/or characterizing at least one host cell protein (HCP) impurity in a sample containing a viral vector. The HCP impurities can be enriched through the utilization of a library of bead-based peptide ligands. The enriched HCPs can subsequently be subjected to enzymatic digestion, generating peptides which can be identified by liquid chromatography-mass spectrometry (LC-MS) analysis to specifically identify and/or characterize said at least one HCP impurity.

### CROSS REFERENCE TO RELATED APPLICATIONS

The present application claims priority to and the benefit of U.S. patent application No. 63/534,637, “HOST CELL PROTEIN ANALYSIS OF AAV USING PROTEOMINER TECHNOLOGY,” which was filed on Aug. 25, 2023, the disclosure of which is hereby incorporated by reference in its entirety.

### SEQUENCE LISTING

The instant application contains a Sequence Listing which is being submitted herewith electronically in XML format and is hereby incorporated by reference in its entirety. Said XML copy, created on Aug. 5, 2024, is named 086939_500701_SL.xml and is 11 kilobytes in size.

### BACKGROUND

Biopharmaceutical products must meet very high standards of purity. Thus, it is important to monitor any impurities in such biopharmaceutical products at different stages of drug development, production, storage and handling. Residual impurities should be at an acceptable low level prior to conducting clinical studies. Residual impurities are also a concern for biopharmaceutical products intended for end-users. For example, host cell proteins (HCPs) can be present in biopharmaceuticals which are developed using cell-based systems. The presence of HCPs in drug products should be monitored and can be unacceptable above a certain threshold, depending on the product and the particular HCP. Sometimes, even trace amounts of HCPs can cause an immunogenic response in an end-user.

Despite significant efforts made to detect HCPs in antibody drugs, information about HCPs in gene therapy products remains limited and has not been widely integrated into host cell engineering or purification processes. HCPs are proteins that are produced by the host organism but remain in drug products (DP). The concentration of HCPs is usually low in the DP after multiple steps of rigid purification applied in the process. However, HCPs are still considered a critical quality attribute (CQA) despite the low abundance due to the potential risk associated with the low abundant residue HCPs, including, but not limited to, immunogenicity, protein clipping and the impact on excipient stability.

Adeno-associated virus (AAV) is the most widely used viral vector for in vivo gene therapy applications. AAVs have low immunogenicity and can enable long-term, stable gene expression. The use of AAVs for gene therapy has created the need for analytical methods to monitor and characterize these products. Process-related and product-related impurities should be monitored to ensure product quality and process consistency.

Monoclonal antibodies (mAbs) have been extensively studied regarding host cell proteins (HCPs). Significant efforts have been devoted to removing, monitoring, identifying, and quantifying HCPs in mAb drug products. Unlike the extensive HCP analysis methods employed for mAb products, the approaches for analyzing HCPs in gene therapy products using mass spectrometry (MS) are limited. Most of HCP identification in gene therapy products have been carried out by direct digestion followed by liquid chromatography-mass spectrometry/mass spectrometry (LC-MS/MS) analysis. SDS-PAGE or 2D-PAGE coupled with in-gel digestion and LC-MS/MS has also been applied to separate HCPs from capsid proteins to allow detection of low abundant HCPs. No enrichment methods have been reported to reduce the dynamic range between AAV capsid protein peptides and HCP peptides.

One of the major challenges for HCP analysis in gene therapy is the limited quantity of product. HCP enrichment methods in mAb drugs usually require at least 1 mg of mAb to accumulate sufficient HCPs for MS detection, however, the quantity of gene therapy product, such as AAV material available for HCP analysis, is usually limited to 10 μg and below. Thus, certain approaches, such as the molecular weight cut-off method, cannot be applied to separate HCPs from AAV capsid proteins because the low abundant HCPs will be mostly adsorbed by the filter with little flowing through the membrane. In addition, Pluronic F-68, a detergent commonly used in AAV products, is also hard to be removed by filtration and the MS signals of detergent will interfere with MS detection of HCP peptides. The anti-HCP polyclonal antibody that can be used to immune-capture HCPs from mAb drugs was proved to be not comprehensive enough to recognize all HCPs from AAV products. In addition, capsid proteins do not contain disulfide bonds, so limiting the amount of trypsin cannot prevent capsid protein from digestion, and therefore HCP analysis in AAV material cannot be improved by limited digestion.

It will be appreciated that a need exists for methods to enrich, identify and characterize HCPs in gene therapy products to monitor and control the residual HCPs in a drug substance or other product to mitigate safety risks.

### SUMMARY

This disclosure provides methods of enriching, identifying and/or characterizing at least one host cell protein (HCP) impurity in a sample containing at least one viral vector, comprising: (a) contacting a sample including at least one HCP impurity to a solid support, wherein said solid support has attached thereto a library of peptide ligands capable of interacting with said at least one HCP impurity generating a slurry; (b) washing said slurry containing at least one HCP impurity to remove unbound material; (c) eluting the bound HCPs to produce an enriched HCP sample; (d) subjecting said enriched HCPs to enzymatic digestion conditions to produce a peptide digest; and (e) subjecting said peptide digest to mass spectrometry/mass spectrometry (MS/MS) analysis to enrich, identify and/or characterize said at least one HCP impurity.

In one aspect, an exemplary solid support with a library of peptide ligands attached thereto are ProteoMiner™ beads.

In one aspect, enriched HCPs are subjected to a denaturant to produce a denatured sample. In a specific aspect, the denaturing agent comprises heat, high pH, low pH, reducing agents, or chaotropic agents.

In one aspect, the enriched HCPs are subjected to an alkylating agent to produce an alkylated sample. In a specific aspect, the alkylating agent comprises iodoacetamide (IOA/IAA), chloroacetamide (CAA), acrylamide (AA), N-ethylmaleimide (NEM), methyl methanethiosulfonate (MMTS), and 4-vinylpyridine or combinations thereof.

In one aspect the enriched HCPs are subjected to a reducing agent to produce a reduced sample. In a specific aspect, the reducing agent comprises dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof.

In one aspect, enriched HCPs are subjected to a denaturant, an alkylating agent and a reducing agent to produce a denatured, reduced and alkylated sample.

In one aspect, the sample contains an AAV vector. In a specific aspect, the AAV vector comprises a serotype selected from a group consisting of AAV1, AAV2, AAV3, AAV4, AAV5, AAV6, AAV7, AAV8, AAVrh8, AAV2/8, AAV9, AAV10, AAV11, AAV12, variations thereof and combinations thereof.

In one aspect, the amount of sample containing at least one host cell protein impurity is about 20 μL to about 100 μL. In a specific aspect, the amount of sample is about 20 μL, about 25 μL, about 30 μL, about 40 μL, about 50 μL, about 60 μL, about 70 μL, about 80 μL, about 90 μL, or about 100 μL, including any and all values in between.

In one aspect, the pH of the binding solution of the interacting peptide ligands is a range of about pH 6.0 to about pH 8.0. In a specific aspect, the pH is about 6.0, about 6.5, about 7.0, about 7.5 or about 8.0.

In one aspect, the volume of interacting peptide ligands is about 0.4 μL to about 15 μL. In a specific aspect, the volume of the interacting peptide ligands is about 0.4 μL, about 1 μL, about 1.5 μL, about 2 μL, about 2.5 μL, about 3 μL, about 4 μL, about 5 μL, about 6 μL, about 7 μL, about 8 μL, about 9 μL, about 10 μL, about 11 μL, about 12 μL, about 13 μL, about 14 μL, about 15 μL or about 20 μL, including any and all values in between.

In one aspect, the digestive enzyme comprises trypsin. In a specific aspect, the enzymatic digestion conditions comprise contacting said denatured, reduced and alkylated sample to at least one digestive enzyme, optionally wherein said at least one digestive enzyme comprises trypsin.

In one aspect, the mass spectrometer is an electrospray ionization mass spectrometer, nano-electrospray ionization mass spectrometer, or a triple quadrupole mass spectrometer.

In one aspect, the mass spectrometer is coupled to a liquid chromatography system.

This disclosure further provides methods of enriching, identifying and/or characterizing at least one host cell protein (HCP) impurity in a sample containing at least one viral vector, comprising: (a) contacting a sample including at least one HCP impurity to a solid support, wherein said solid support has attached thereto a library of peptide ligands capable of interacting with said at least one HCP impurity generating a slurry; (b) washing said slurry containing at least one HCP impurity to remove unbound material; (c) eluting bound HCPs to produce an enriched HCP sample; (d) subjecting said enriched HCP sample to a denaturant to produce a denatured sample; (e) subjecting said denatured sample to a reducing agent to produce a denatured and reduced sample; (f) subjecting said denatured and reduced sample to an alkylating agent to produce a denatured, reduced and alkylated sample; (g) subjecting said denatured, reduced and alkylated sample to enzymatic digestion conditions to produce a peptide digest; and (h) subjecting said peptide digest to liquid chromatography-mass spectrometry/mass spectrometry (LC-MS/MS) analysis to identify and/or characterize said at least one HCP impurity.

In one aspect, the sample contains an AAV vector. In a specific aspect, the AAV vector comprises a serotype selected from a group consisting of AAV1, AAV2, AAV3, AAV4, AAV5, AAV6, AAV7, AAV8, AAVrh8, AAV2/8, AAV9, AAV10, AAV11, AAV12, variations thereof and combinations thereof.

In one aspect, the amount of sample containing at least one host cell protein impurity is about 20 μL to about 100 μL. In a specific aspect, the amount of sample is about 20 μL, about 25 μL, about 30 μL, about 40 μL, about 50 μL, about 60 μL, about 70 μL, about 80 μL, about 90 μL, or about 100 μL, including any and all values in between.

In one aspect, the volume of interacting peptide ligands is about 0.4 μL to about 15 μL. In a specific aspect, the volume of the interacting peptide ligands is about 0.4 μL, about 1 μL, about 1.5 μL, about 2 μL, about 2.5 μL, about 3 μL, about 4 μL, about 5 μL, about 6 μL, about 7 μL, about 8 μL, about 9 μL, about 10 μL, about 11 μL, about 12 μL, about 13 μL, about 14 μL, about 15 μL or about 20 μL, including any and all values in between.

In one aspect, enriched HCPs are subjected to a denaturant to produce a denatured sample. In a specific aspect, the denaturing agent comprises heat, high pH, low pH, reducing agents, or chaotropic agents.

In one aspect, the enriched HCPs are subjected to an alkylating agent to produce an alkylated sample. In a specific aspect, the alkylating agent comprises iodoacetamide (IOA/IAA), chloroacetamide (CAA), acrylamide (AA), N-ethylmaleimide (NEM), methyl methanethiosulfonate (MMTS), and 4-vinylpyridine or combinations thereof.

In one aspect the enriched HCPs are subjected to a reducing agent to produce a reduced sample. In a specific aspect, the reducing agent comprises dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof.

In one aspect, the digestive enzyme comprises trypsin. In a specific aspect, the enzymatic digestion conditions comprise contacting said denatured, reduced and alkylated sample to at least one digestive enzyme, optionally wherein said at least one digestive enzyme comprises trypsin.

In one aspect, the mass spectrometer is an electrospray ionization mass spectrometer, nano-electrospray ionization mass spectrometer, or a triple quadrupole mass spectrometer.

In one aspect, the mass spectrometer is coupled to a liquid chromatography system.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. In the drawings:

FIG. 1A shows average HCPs detected by an enrichment method of the present disclosure using varying concentrations of ProteoMiner™ beads as compared to direct digestion, according to an exemplary embodiment.

FIG. 1B shows the number of unique peptides detected by an enrichment method of the present disclosure using varying concentrations of ProteoMiner™ beads as compared to direct digestion, according to an exemplary embodiment.

FIG. 1C shows the number of HCPs identified using varying sample volume,

according to an exemplary embodiment.

FIG. 1D shows the number of unique peptides identified using varying sample volume, according to an exemplary embodiment.

FIG. 1E shows the number of HCPs identified by an enrichment method of the present disclosure at varying pH as compared to direct digestion, according to an exemplary embodiment.

FIG. 1F shows the number of HCPs identified by an enrichment method of the present disclosure of several AAV serotypes at varying pH, according to an exemplary embodiment.

FIG. 2A shows the number of HCPs identified from an AAV8 full capsid sample identified by an enrichment method of the present disclosure compared with direct digestion, according to an exemplary embodiment.

FIG. 2B shows the number of HCPs identified from an AAV8 full capsid sample identified by an enrichment method of the present disclosure across triplicate replicates, according to an exemplary embodiment.

FIG. 3A shows the number of HCPs identified from several empty capsid AAV subtypes utilizing an enrichment method of the present disclosure compared with direct digestion, according to an exemplary embodiment.

FIG. 3B shows a Venn diagram of the HCPs identified from several empty capsid AAV subtypes utilizing an enrichment method of the present disclosure compared with direct digestion, according to an exemplary embodiment.

FIG. 4 shows a comparison of the percentage of previously reported HCPs using ProteoMiner™ compared to the percentage of HCPs detected by an enrichment method of the present disclosure, according to an exemplary embodiment.

FIG. 5A shows the quantitation of two different peptides, VFEHIGK (SEQ ID NO: 1) and NVAVDELSR (SEQ ID NO: 2), before and after an enrichment method of the present disclosure, according to an exemplary embodiment.

FIG. 5B shows the MS2 spectrum of two low abundance peptides, EIEIDIEPTDKVER (SEQ ID NO: 3) and TAFQEALDAAGDK (SEQ ID NO: 4), utilizing an enrichment method of the present disclosure, according to an exemplary embodiment.

FIG. 6 shows a comparison of a TIC chromatogram of a sample prepared by direct digestion as compared to an AAV product prepared by on-filter digestion, according to an exemplary embodiment.

FIG. 7A shows a TIC chromatography of sample prepared by direct digestion.

FIG. 7B is a corresponding MS spectrum (of FIG. 7A) of the sample prepared by direct digestion. MS spectrum was collected from retention time from 50 to 81 min.

FIG. 7C shows a TIC chromatography of sample prepared by ProteoMiner™ enrichment method.

FIG. 7D is a corresponding MS spectrum (of FIG. 7C) of the sample prepared by the ProteoMiner™ enrichment method. MS spectrum was collected from retention time from 50 to 90 min.

FIG. 8 is a schematic of a method of the present disclosure, according to an embodiment.

### DETAILED DESCRIPTION

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any sub-combination.

The present disclosure provides methods of enriching, identifying and/or characterizing at least one host cell protein (HCP) impurity in a sample containing at least one viral vector, comprising: (a) contacting a sample including at least one HCP impurity to a solid support, wherein said solid support has attached thereto a library of peptide ligands capable of interacting with said at least one HCP impurity generating a slurry; (b) washing said slurry containing at least one HCP impurity to remove unbound material; (c) eluting the bound HCPs to produce an enriched HCP sample; (d) subjecting said enriched HCPs to enzymatic digestion conditions to produce a peptide digest; and (c) subjecting said peptide digest to mass spectrometry/mass spectrometry (MS/MS) analysis to enrich, identify and/or characterize said at least one HCP impurity.

The present disclosure provides methods of enriching, identifying and/or characterizing at least one host cell protein (HCP) impurity in a sample containing at least one viral vector, comprising: (a) contacting a sample including at least one HCP impurity to a solid support, wherein said solid support has attached thereto a library of peptide ligands capable of interacting with said at least one HCP impurity generating a slurry; (b) washing said slurry containing at least one HCP impurity to remove unbound material; (c) eluting bound HCPs to produce an enriched HCP sample; (d) subjecting said enriched HCP sample to a denaturant to produce a denatured sample; (e) subjecting said denatured sample to a reducing agent to produce a denatured and reduced sample; (f) subjecting said denatured and reduced sample to an alkylating agent to produce a denatured, reduced and alkylated sample; (g) subjecting said denatured, reduced and alkylated sample to enzymatic digestion conditions to produce a peptide digest; and (h) subjecting said peptide digest to liquid chromatography-mass spectrometry/mass spectrometry (LC-MS/MS) analysis to identify and/or characterize said at least one HCP impurity.

In some embodiments, said enriched HCPs are subjected to a denaturant to produce a denatured sample.

In some embodiments, said denaturant comprises heat, high pH, low pH, reducing agents, or chaotropic agents.

In some embodiments, said enriched HCPs are subjected to an alkylating agent to produce an alkylated sample.

In some embodiments, said alkylating agent comprises iodoacetamide (IOA/IAA), chloroacetamide (CAA), acrylamide (AA), N-ethylmaleimide (NEM), methyl methanethiosulfonate (MMTS), 4-vinylpyridine, or combinations thereof.

In some embodiments, said enriched HCPs are subjected to a reducing agent to produce a reduced sample.

In some embodiments, said reducing agent comprises dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof.

In some embodiments, said enriched HCPs are subjected to a denaturant, an alkylating agent, and a reducing agent to produce a denatured, reduced, and alkylated sample.

In some embodiments, the viral vector is an AAV vector.

In some embodiments, said AAV vector comprises a serotype selected from AAV1, AAV2, AAV3, AAV4, AAV5, AAV6, AAV7, AAV8, AAVrh8, AAV2/8, AAV9, AAV10, AAV11, AAV12, variations thereof and combinations thereof.

In some embodiments, the amount of sample containing at least one host cell protein impurity is about 20 μL to about 100 μL.

In some embodiments, the library of peptide ligands comprises a binding solution with a pH of about pH 6.0 to about pH 8.0, optionally wherein said pH is about 7.0.

In some embodiments, the volume of the library of peptide ligands is about 0.4 μL to about 15 μL.

In some embodiments, said enzymatic digestion conditions comprise contacting said denatured, reduced and alkylated sample with at least one digestive enzyme.

In some embodiments, said at least one digestive enzyme comprises trypsin.

In some embodiments, said mass spectrometer is an electrospray ionization mass spectrometer, nano-electrospray ionization mass spectrometer, or a triple quadrupole mass spectrometer. In some embodiments, said mass spectrometry system is electrospray ionization mass spectrometer, nano-electrospray ionization mass spectrometer, or an Orbitrap-based mass spectrometer. In some embodiments, said mass spectrometer is coupled to a liquid chromatography system.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

In order to manufacture biopharmaceutical products, it is important to obtain biopharmaceutical products having high purity, since residual HCPs can compromise product safety and stability. Several methods have been developed for characterizing and identifying HCPs in mAbs, including non-mass spectrometry (MS) based techniques such as enzyme-linked immunosorbent assay (ELISA) and gel-based analysis, as well as MS-based methods. The analysis of HCPs in mAb drugs using mass spectrometry (MS) has undergone rapid development and is increasingly being employed for HCP characterization. This is primarily due to the high sensitivity and the capability to identify individual HCPs, which greatly aids in risk assessment efforts. The key to develop a sensitive MS method for HCP analysis is to reduce the dynamic range between mAb peptides and HCP peptides. Various approaches have been utilized to achieve this goal. The approaches include depleting mAb using Protein A affinity column (Protein A depletion), precipitating mAb by insufficiently digest antibody (limited digestion), enrichment of HCPs using anti-HCP antibody (ELISA-immunocapture coupled with LC-MS/MS), enriching HCPs by removing mAbs using molecular weight cut off filter (filtration) or size-exclusion chromatography (SEC), enriching HCPs using ProteoMiner™ beads with or without coupling with limited digestion (PMLD or PM).

ProteoMiner™ beads are a library of bead-based peptide ligands that can bind to diverse types of proteins.

Adeno-associated viruses (AAVs) have been widely used as gene delivery vectors to deliver genetic material, such as delivering nucleic acids for gene therapy. AAVs provide the advantages of non-pathogenicity and low immunogenicity. AAVs are nonpathogenic members of the Parvoviridae family under Dependovirus genus and require helpers, such as Adenovirus or Herpesvirus, for infection (Venkatakrishnan et al., Structure and Dynamics of Adeno-Associated Virus Serotype 1 VP1-Unique N-Terminal Domain and Its Role in Capsid Trafficking, Journal of Virology, May 2013, vol. 87, no. 9, pages 4974-4984). AAV encapsulates a single-stranded DNA genome of about 4.8 kilobases (kb) in an icosahedral capsid which is made of a shell of capsid viral proteins. Recombinant AAV genomes are nonpathogenic and do not integrate into a host's genome, but exist as stable episomes that provide long-term expression. AAV serotypes make a very useful system for preferentially transducing specific cell types.

Overall, AAV-based therapy has the advantages of being non-pathogenic and non-toxic, having cell type-specific infection, and offering different serotypes with varying cell transduction efficiencies. A disadvantage is that AAV production, purification, and characterization are more complex compared to, for example, antibody therapies. Fully packaged AAVs consist of an icosahedral capsid containing an about 4.8 kb single-stranded genome. An empty capsid has a molecular weight of about 3750 kDa, while a full capsid with an about 4.7 kb single-stranded genome has a molecular weight of about 5100 kDa. The purity of AAVs is defined by several product-related impurities, including empty capsids, capsids containing partial or incorrect genomes, and aggregated or degraded capsid, as well as residual HCPs.

In order to manufacture biopharmaceutical products, it is important that such products have high purity, since residual HCPs can compromise product safety and stability. The characterization of HCPs in viral vector production has posed many challenges. For example, the location of the HCPs must be considered. HCPs may be located inside the vector, associating with the target genome, and/or part of the capsid of the AAV. In addition, the AAV serotype may have a profound effect on the HCPs identified.

Huang et al. (Huang et al., “A Novel Sample Preparation for Shotgun Proteomics Characterization of HCPs in Antibodies,” Anal. Chem. 2017 May 16; 89 (10): 5436-5444) describes a sample preparation method using trypsin digestion for shotgun proteomics characterization of HCP impurities in an antibody sample. Huang's sample preparation method maintains the antibody nearly intact while HCPs are digested. Huang's approach can reduce the dynamic range for HCP detection using mass spectrometry by one to two orders of magnitude compared to traditional trypsin digestion sample preparation. As demonstrated by HCP spiking experiments, Huang's approach can detect 0.5 ppm of HCPs with molecular weight greater than 60 kDa, such as rPLBL2. For example, sixty mouse HCP impurities were detected in RM 8670 (NISTmAb, NIST monoclonal antibody standard, expressed in a murine cell line, obtained from the National Institute of Standards and Technology, Gaithersburg, MD) using Huang's approach.

Doncanu et al. (Doncanu et al., “Enhanced Detection of Low-Abundance Host Cell Protein Impurities in High-Purity Monoclonal Antibodies Down to 1 ppm Using Ion Mobility Mass Spectrometry Coupled with Multidimensional Liquid Chromatography,” Anal. Chem. 2015 Oct. 20; 87(20):10283-10291) reports the detection of low-abundance HCP impurities down to 1 ppm in antibody samples using liquid chromatography-mass spectrometry (LC-MS) methods. Doneanu's approach includes using a new charge-surface-modified C18 stationary phase to mitigate the challenges of column saturation, incorporating traveling-wave ion mobility separation of co-eluting peptide precursors, and improving fragmentation efficiency of low-abundance HCP peptides by correlating the collision energy used for precursor fragmentation with the mobility drift time. HCP impurities can be identified at 10-50 ppm using 2D-HPLC (2D-High Performance Liquid Chromatography) in combination with ion mobility mass spectrometry analysis. However, the cycle times for 2D-LC or 2D-HPLC can be very long. In addition, these methods may not be sensitive enough for low level HCP analysis, such as less than 10 ppm. Other approaches of identifying HCP impurities include sample preparations to enrich HCPs by removing antibodies in the sample, such as using affinity purification or limited digestion to remove antibodies. In addition, using polyclonal antibodies to capture HCPs is another common approach.

Analytical techniques required for identifying HCP impurities encounter the challenges of dealing with about 1 million times more matrix molecules than the analytes, for example, HCPs or HCP peptides, due to very high sample complexity. Enriching HCPs to levels compatible with detection is difficult, since HCP impurities are most often present at low levels, such as 1-100 ppm, in protein biopharmaceuticals. Without knowing the identities and properties of HCPs, it can be very challenging to develop a general sample preparation procedure to enrich HCPs (or HCP peptides) or remove the matrix background (Doncanu et al.).

Chen et al. (Chen et al., “Improved host cell protein analysis in monoclonal antibody products through ProteoMiner,” Anal. Biochem. 2020 Dec. 1; 610:113972) describes a method of enriching HCPs using interacting peptide ligands, particularly ProteoMiner™ beads. The method of the present disclosure improves upon the previously described ProteoMiner™ method of HCP enrichment, identification and quantification.

The present application provides methods to enrich HCPs using interacting peptide ligands, such as a combinatorial ligand library. In some exemplary embodiments, ProteoMiner™ beads (Bio-Rad Laboratories, Inc., Hercules, CA), a combinatorial hexapeptide library immobilized on beads, are used to enrich HCPs. When the peptide ligand-conjugated beads are applied to a sample containing various protein species, each protein species can bind to its interacting peptide ligands. HCPs bind to their interacting peptide ligands mainly by hydrophobic force in combination with some weak interaction forces, such as ionic interaction and hydrogen bonding.

A protein species that is in high abundance can saturate its interacting peptide ligands due to the presence of excess quantity, since there are limited numbers of interacting peptide ligands corresponding to each protein species in the combinatorial ligand library. The limited numbers of corresponding interacting peptide ligands can be saturated easily in the presence of excess quantity of high-abundance proteins. The excess quantity of high-abundance proteins that are unable to bind to the interacting peptide ligands can be washed off from the beads. Since the quantity of low-abundance proteins in the sample is relatively low in comparison to the high-abundance proteins, the low-abundance proteins may not saturate their corresponding interacting peptide ligands. Therefore, the low-abundance proteins can be relatively enriched in comparison to the high-abundance proteins. After conducting the enrichment process, the broad dynamic range of protein concentrations can be significantly reduced to allow detection of low abundance proteins.

In order to identify HCPs, several approaches have been employed, such as gel electrophoresis and/or digestion coupled with liquid chromatography-mass spectrometry (LC-MS), or digestion followed by enrichment with liquid chromatography (LC) followed by LC-MS. In addition, the use of direct digestion, immunoprecipitation, native digestion and molecular weight cut off filtration techniques have been implemented. However, the dynamic concentration and location of HCPs in the AAVs and the limitations of sample size and AAV concentration are major challenges to monitor and remove HCP impurities. In order to overcome this issue, methods of enriching, identifying and characterizing HCPs have been developed.

The present application provides HCP enrichment, identification and characterization methods for gene therapy products, and specifically AAV products, using ProteoMiner™ enrichment beads. In some exemplary embodiments, the method improved the dynamic range of HCP detection by one to two orders of magnitudes compared to direct digestion and allowed 5-fold to 10-fold increase of the number of HCPs detected from AAV material. In addition, detergent was found to be completely removed with no interference on detection of HCP peptides. This approach can be applied to other gene therapy products and can be used to troubleshoot and identify potential problematic HCPs for risk assessment.

This disclosure provides methods to satisfy the aforementioned demands by providing methods to identify HCPs in a biopharmaceutical product to mitigate safety risks. Exemplary embodiments disclosed herein satisfy the aforementioned demands and the long-felt needs.

Unless described otherwise, all technical and scientific terms used herein have the same meaning as commonly understood by one of ordinary skill in the art to which this disclosure belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule.

As used herein, the term “therapeutic protein” includes any of proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies.

In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., 28 BIOTECHNOLOGY AND GENETIC ENGINEERING REVIEWS 147-176 (2012), the entirety of which is herein incorporated by reference). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the present disclosure, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” (bsAbs) includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with cach heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or kλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entirety of which is herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, the term “multispecific antibody” refers to an antibody with binding specificities for at least two different antigens. While such molecules normally will only bind two antigens (e.g., bispecific antibodies, bsAbs), antibodies with additional specificities such as trispecific antibody and KIH Trispecific are also contemplated.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies can be prepared using a wide variety of techniques known in the art including the use of hybridoma, recombinant, and phage display technologies, or a combination thereof.

As used herein, a “protein pharmaceutical product” or “biopharmaceutical product” includes an active ingredient which can be fully or partially biological in nature. In one aspect, the protein pharmaceutical product can comprise a peptide, a protein, a fusion protein, an antibody, an antigen, vaccine, a peptide-drug conjugate, an antibody-drug conjugate, a protein-drug conjugate, cells, tissues, or combinations thereof. In another aspect, the protein pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate, cells, tissues, or combinations thereof.

As used herein, a “sample” refers to a mixture of molecules that comprises at least a viral particle, such as an AAV particle, or an empty viral capsid, that is subjected to manipulation in accordance with the methods of the disclosure, including, for example, separating, analyzing, extracting, concentrating, profiling and the like. The sample can be obtained from any step of a bioprocess, such as cell culture fluid (CCF), harvested cell culture fluid (HCCF), any step in the downstream processing, drug substance (DS), or a drug product (DP) comprising the final formulated product. In some specific exemplary embodiments, the sample can be selected from any step of the downstream process of clarification, chromatographic production, or filtration. In some specific exemplary embodiments, the drug product can be selected from manufactured drug product in the clinic, shipping, storage, or handling.

As used herein, the term “impurity” can include any undesirable protein present in a protein sample or protein biopharmaceutical product. Impurities can include process and product-related impurities. Impurities can include non-protein molecules including, but not limited to, chemical and biochemical processing reagents, inorganic salts, solvents, carriers, and other leachables. The impurity can further be of known structure, partially characterized, or unidentified. Process-related impurities can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

As used herein, the term “host-cell protein” (HCP) includes protein derived from the host cell. Host-cell protein can be a process-related impurity which can be derived from the manufacturing process and can include three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables. In some exemplary embodiments, the types of HCP process-related impurities in the composition can be at least two.

The presence of a host cell protein in a biotherapeutic product may be considered to be a higher or lower risk based on a number of measurable factors. One such factor is the concentration or abundance (quantity) of an HCP impurity in a biotherapeutic product. An HCP may have no discernible impact at a low enough abundance, as measured by, for example, ELISA or mass spectrometry. The level at which an HCP may present a considerable risk, which may be considered an unacceptable level in a product and may be monitored as a critical quality attribute (CQA), may depend on the specific identity of the HCP. Particular HCPs may be known to present a risk at a particular level, for example depending on the level of enzymatic activity of an HCP that is an enzyme.

Relatedly, the criticality of the presence of an HCP may depend on the function of that HCP, in particular in relation to the components of the biotherapeutic product. For example, an HCP lipase that may or is known to degrade polysorbate that is present in the biotherapeutic product of interest may be closely monitored and may have a low threshold for how much of the HCP impurity can be allowed in the biotherapeutic product. Other HCPs of particular concern may be, for example, proteases that may or are known to degrade a protein of interest in the biotherapeutic product, or immunogenic HCPs that may or are known to cause an immune reaction when administered to a subject. Using the method of the present disclosure, a person skilled in the art may evaluate the abundance, distribution, and/or identity of an HCP impurity in the context of the biotherapeutic product of interest to determine if the HCP impurity is an HCP impurity of concern, and based on that determination may use chromatographic or other separation methods to remove the impurity when producing the biotherapeutic product.

In some exemplary embodiments, a sample can comprise at least one high-abundance protein or peptide and at least one HCP. In some exemplary embodiments, a concentration of the at least one high-abundance protein or peptide can be at least about 1000 times, about 10,000 times, about 100,000 times or about 1,000,000 times higher than a concentration of the at least one HCP. Another way of expressing the relative concentrations is, for example, in parts per million (ppm). It should be understood that when using ppm to describe the concentration of a low-abundance protein or peptide, such as an HCP, in a sample that includes a high-abundance protein or peptide, such as a therapeutic protein, ppm is measured relative to the concentration of the high-abundance protein or peptide. In some exemplary embodiments, a concentration of the at least one HCP can be less than about 1000 ppm, less than about 100 ppm, less than about 10 ppm, or less than about 1 ppm.

The terms “peptide,” “protein” and “polypeptide” refer, interchangeably, to a polymer of amino acids and/or amino acid analogs that are joined by peptide bonds or peptide bond mimetics. The twenty naturally-occurring amino acids and their single-letter and three-letter designations are as follows: Alanine A Ala; Cysteine C Cys; Aspartic Acid D Asp; Glutamic acid E Glu; Phenylalanine F Phe; Glycine G Gly; Histidine H His; Isoleucine I He; Lysine K Lys; Leucine L Leu; Methionine M Met; Asparagine N Asn; Proline P Pro; Glutamine Q Gln; Arginine R Arg; Serine S Ser; Threonine T Thr; Valine V Val; Tryptophan w Trp; and Tyrosine Y Tyr.

A “vector,” as used herein, refers to a recombinant plasmid or virus (“viral vector”) that comprises a nucleic acid to be delivered into a host cell, either in vitro or in vivo. Vectors derived from AAV are particularly attractive for delivering genetic material because (i) they are able to infect (transduce) a wide variety of non-dividing and dividing cell types including muscle fibers and neurons; (ii) they are devoid of the virus structural genes, thereby eliminating the natural host cell responses to virus infection, for example, interferon-mediated responses; (iii) wild type AAVs have never been associated with any pathology in humans; (iv) in contrast to wild type AAVs, which are capable of integrating into the host cell genome, replication-deficient AAV vectors generally persist as episomes, thus limiting the risk of insertional mutagenesis or activation of oncogenes; and (v) in contrast to other vector systems, AAV vectors do not trigger a significant immune response (see ii), thus granting long-term expression of the therapeutic transgenes (provided their gene products are not rejected).

A “recombinant viral vector” refers to a recombinant polynucleotide vector including one or more heterologous sequences (e.g., nucleic acid sequence not of viral origin).

A “recombinant AAV vector (rAAV vector)” refers to a polynucleotide vector including one or more heterologous sequences (e.g., nucleic acid sequence not of AAV origin) that may be flanked by at least one, e.g., two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (e.g. AAV Rep and Cap proteins).

A “viral particle” refers to a particle composed of at least one viral capsid protein and an encapsulated viral genome. While AAV is described in this disclosure as a model virus or viral particle, it is contemplated that the disclosed methods can be applied to profile a variety of viruses, e.g., the viral families, subfamilies, and genera. In some aspects, the viral capsid, virus, or viral particle belongs to a viral family selected from the group consisting of Adenoviridae, Parvoviridae, Retroviridae, Baculoviridae, and Herpesviridae. In some aspects, the viral capsid, virus, or viral particle belongs to a viral genus selected from the group consisting of Atadenovirus, Aviadenovirus, Ichtadenovirus, Mastadenovirus, Siadenovirus, Ambidensovirus, Brevidensovirus, Hepandensovirus, Iteradensovirus, Penstyldensovirus, Amdoparvovirus, Aveparvovirus, Bocaparvovirus, Copiparvovirus, Dependoparvovirus, Erythroparvovirus, Protoparvovirus, Tetraparvovirus, Alpharetrovirus, Betaretrovirus, Deltaretrovirus, Epsilonretrovirus, Gammaretrovirus, Lentivirus, Spumavirus, Alphabaculovirus, Betabaculovirus, Deltabaculovirus, Gammabaculovirus, Iltovirus, Mardivirus, Simplexvirus, Varicellovirus, Cytomegalovirus, Muromegalovirus, Proboscivirus, Roscolovirus, Lymphocryptovirus, Macavirus, Percavirus, and Rhadinovirus.

A “capsid” is the protein shell of a virus, which encloses the genetic material. Three viral capsid proteins, VP1, VP1 and VP3, form the viral icosahedral capsid of 60 subunits in a ratio of 1:1:10. A full capsid contains genetic material and is required to provide therapeutic benefit. An empty capsid lacks the genome and therefore lacks the ability to provide therapeutic benefit to the patient.

As used herein, the term “gene therapy” is a method of treatment of a genetic disease by modifying or manipulating a gene of interest. The key step in gene therapy is efficient delivery of the vector to the appropriate tissue or cells. Non-limiting examples of gene therapy products can include plasmid DNA, viral vectors, non-viral vectors, bacterial vectors, human gene editing technology, and patient-derived cellular gene therapy products.

As used herein, the term “solid support” can include any surface with an ability to bind a protein or peptide. Non-limiting examples of solid supports can include affinity resins, beads and coated plates or microplates. Solid supports can be attached to molecules capable of binding to a protein or peptide, including affinity reagents, antigen-binding molecules, or interacting peptide ligands. In some exemplary embodiments, a solid support comprises beads attached to interacting peptide ligands. In some exemplary embodiments, a solid support comprises ProteoMiner™ beads.

In some exemplary embodiments, the sample can be prepared prior to LC-MS analysis. Preparation steps can include reduction, denaturation, alkylation, dilution, digestion, and separation (for example, centrifugation).

As used herein, “protein denaturing” or “denaturation” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT, or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples of chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

Proteins may have distinctive susceptibility to denaturation. For example, viral proteins assembled into a viral capsid may have reduced susceptibility to denaturation compared to a monomeric or smaller multimeric protein, for example a host cell protein. A difference in susceptibility to denaturation may be taken advantage of in order to preferentially denature a particular protein or class of proteins while leaving another protein or class of proteins in a substantially natively folded state. In some aspects, denaturation may be performed at a temperature selected to substantially denature a protein or class of proteins, for example non-viral proteins such as host cell proteins, while leaving the proteins of a viral capsid substantially in a folded state. This may be referred to as, for example, mild denaturation, limited denaturation, partial denaturation, or differential denaturation. A partially denatured sample may then be subjected to a digestion step to produce a peptide digest. Because denatured proteins are more susceptible to digestion by digestive enzymes, the peptide digest will preferentially include peptides from the more denatured proteins, for example non-viral proteins such as HCPs, compared to peptides from the more natively folded proteins, for example viral capsid proteins. This may be referred to as, for example, partial digestion, differential digestion, or limited digestion. The peptide digest will be enriched for peptides of the non-viral protein, for example HCPs, relative to the original sample. This enrichment may be useful for subsequent analysis, for example liquid chromatography-mass spectrometry analysis, in order to sensitively and accurately identify, characterize, and quantify the non-viral proteins.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion. Digestion of a protein into constituent peptides can produce a “peptide digest” that can further be analyzed using peptide mapping analysis.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, 12 JOURNAL OF PROTEOME RESEARCH 1067-1077 (2013)).

Conventional methods use a digestive enzyme in conditions and concentrations sufficient to completely digest all protein in a sample prior to LC-MS analysis. The present disclosure surprisingly finds that identification and quantification of low-abundance proteins such as HCPs can be improved through limited digestion, meaning that denaturation and digestion conditions are selected such that proteins in a sample are not completely digested. In some exemplary embodiments, proteins are subjected to mild denaturation or partial denaturation prior to digestion, such that a particular protein or class of proteins that are more susceptible to denaturation are preferentially digested.

As used herein, the term “protein reducing agent” or “reduction agent” refers to the agent used for reduction of disulfide bridges in a protein. Non-limiting examples of protein reducing agents used to reduce a protein are dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof. A reducing step may be performed in sequence with or concurrent with other sample preparation steps. For example, a reducing step and a denaturing step may be performed simultaneously (by adding a reducing agent while incubating a sample at high temperatures) so that cysteines exposed to the solvent by denaturing can be accessed by the reducing agent.

As used herein, the term “protein alkylating agent” or “alkylation agent” refers to an agent used for alkylating certain free amino acid residues in a protein. Non-limiting examples of protein alkylating agents are iodoacetamide (IOA/IAA), chloroacetamide (CAA), acrylamide (AA), N-ethylmaleimide (NEM), methyl methanethiosulfonate (MMTS), and 4-vinylpyridine or combinations thereof.

As used herein, the term “liquid chromatography” refers to a process in which a biological and/or chemical mixture carried by a liquid can be separated into components as a result of differential distribution of the components as they flow through (or into) a stationary liquid or solid phase. Non-limiting examples of liquid chromatography include reverse phase liquid chromatography, ion-exchange chromatography, size exclusion chromatography, affinity chromatography, hydrophobic interaction chromatography, hydrophilic interaction chromatography, or mixed-mode chromatography. In some aspects, the sample or eluate can be subjected to any one of the aforementioned chromatographic methods or a combination thereof.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

The mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion was selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

SRM/MRM/Selected-ion monitoring (SIM) is a method used in tandem mass spectrometry in which an ion of a particular mass is selected in the first stage of a tandem mass spectrometer and an ion product of a fragmentation reaction of the precursor ion is selected in the second mass spectrometer stage for detection. Examples of triple quadrupole mass spectrometers (TQMS) that can perform MRM/SRM/SIM include but are not limited to QTRAP® 6500 System (Sciex), QTRAP® 5500 System (Sciex), Triple QTriple Quad 6500 System (Sciex), Agilent 6400 Series Triple Quadrupole LC/MS systems, and Thermo Scientific™ TSQ™ Triple Quadrupole system.

In addition to MRM, the choice of peptides can also be quantified through Parallel-Reaction Monitoring (PRM). PRM is the application of SRM with parallel detection of all transitions in a single analysis using a high-resolution mass spectrometer. PRM provides high selectivity, high sensitivity and high-throughput to quantify selected peptides (Q1), and hence quantify proteins. Multiple peptides can be specifically selected for each protein. PRM methodology can use the quadrupole of a mass spectrometer to isolate a target precursor ion, fragment the targeted precursor ion in the collision cell, and then detect the resulting product ions in the Orbitrap mass analyzer. PRM can use a quadrupole time-of-flight (QTOF) or hybrid quadrupole-orbitrap (QOrbitrap) mass spectrometer to carry out the identification of peptides and/or proteins. Examples of QTOF include but are not limited to TripleTOF® 6600 System (Sciex), TripleTOF® 5600 System (Scicx), X500R QTOF System (Sciex), 6500 Series Accurate-Mass Quadrupole Time-of-Flight (Q-TOF) (Agilent) and Xevo G2-XS QT of Quadrupole Time-of-Flight Mass Spectrometry (Waters). Examples of QObitrap include but are not limited to Q Exactive™ Hybrid Quadrupole-Orbitrap Mass Spectrometer (Thermo Scientific) and Orbitrap Fusion™ Tribrid™ (Thermo Scientific).

Non-limiting advantages of PRM include: elimination of most interferences; providing more accuracy and attomole-level limits of detection and quantification; enabling the confident confirmation of the peptide identity with spectral library matching; reducing assay development time since no target transitions need to be preselected; and ensuring UHPLC-compatible data acquisition speeds with spectrum multiplexing and advanced signal processing.

The mass spectrometer in the methods or systems of the present application can be, for example, an electrospray ionization mass spectrometer, nano-electrospray ionization mass spectrometer, or a triple quadrupole mass spectrometer, wherein the mass spectrometer can be coupled to a liquid chromatography system, wherein the mass spectrometer is capable of performing LC-MS (liquid chromatography-mass spectrometry) or LC-PRM-MS (liquid chromatography-parallel reaction monitoring-mass spectrometry) analyses. In some exemplary embodiments, the identification of peptides is performed using PRM-MS.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. MS/MS, or MS2, can be performed by first selecting and isolating a precursor ion (MS1), and fragmenting it to obtain meaningful information. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited to, identifying the protein, sequencing amino acids of the protein fragments, determining protein sequencing, quantifying the protein, locating post-translational modifications, identifying post translational modifications, or comparability analysis, or combinations thereof.

In some exemplary aspects, the mass spectrometer can use nanoelectrospray or nanospray ionization. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (matrixscience.com), Spectrum Mill (chem.agilent.com), PLGS (waters.com), PEAKS (bioinformaticssolutions.com), Proteinpilot (download.appliedbiosystems.com/proteinpilot), Phenyx (phenyx-ms.com), Sorcerer (sagenresearch.com), OMSSA (pubchem.ncbi.nlm.nih.gov/omssa/), X!Tandem (thegpm.org/TANDEM/), Protein Prospector (prospector.ucsf.edu/prospector/mshome.htm), Byonic (proteinmetrics.com/products/byonic) or Sequest (fields.scripps.edu/sequest).

An alternative embodiment of the present disclosure includes methods of enriching identifying and/or characterizing at least one host cell protein impurity in a sample containing at least one mAb or protein comprising contacting a sample including at least one HCP impurity to a solid support, wherein said solid support is attached to interacting peptide ligands capable of interacting with said at least one HCP impurity generating a slurry, washing said slurry of beads containing at least one HCP impurity to remove unbound material, eluting the bound HCPs to produce an enriched HCP sample, subjecting said enriched HCPs to enzymatic digestion conditions to produce a peptide digest and subjecting said peptide digest to mass spectrometry/mass spectrometry (MS/MS) analysis to enrich, identify and/or characterize said at least one HCP impurity.

It is understood that the present disclosure is not limited to any of the aforesaid protein(s), therapeutic protein(s), antibody(s), recombinant protein(s), host-cell protein(s), protein pharmaceutical product(s), sample(s), AAV(s), virus(es), serotype(s), vector(s), protein alkylating agent(s), protein denaturing agent(s), protein reducing agent(s), digestive enzyme(s), chromatographic method(s), mass spectrometer(s), database(s), bioinformatics tool(s), pH range(s) or value(s), temperature(s), or concentration(s), and any protein(s), therapeutic protein(s), antibody(s), recombinant protein(s), host-cell protein(s), protein pharmaceutical product(s), sample(s), AAV(s), virus(es), serotype(s), vector(s), protein alkylating agent(s), protein denaturing agent(s), protein reducing agent(s), digestive enzyme(s), chromatographic method(s), mass spectrometer(s), database(s), bioinformatics tool(s), pH, temperature(s), or concentration(s) can be selected by any suitable means.

The present disclosure will be more fully understood by reference to the following examples. They should not, however, be construed as limiting the scope of the disclosure.

Sequence Table

| Sequence | SEQ ID NO |
|---|---|
| VFEHIGK | 1 |
| NVAVDELSR | 2 |
| EIEIDIEPTDKVER | 3 |
| TAFQEALDAAGDK | 4 |
| NTPVPADPPTTFNQSK | 5 |
| LELLAQR | 6 |
| NSLANPGIAMATHKDDEER | 7 |
| HPPPQILIK | 8 |
| VSTTTGQNNNSNFAWTAGTK | 9 |
| LVNEVTEFAK | 10 |

### EMBODIMENTS

The following list of embodiments is intended to complement, rather than displace or supersede, the previous descriptions.
Embodiment 1. A method of enriching, identifying and/or characterizing at least one host cell protein (HCP) impurity in a sample containing at least one viral vector, comprising:
(a) contacting a sample including at least one HCP impurity to a solid support, wherein said solid support has attached thereto a library of peptide ligands capable of interacting with said at least one HCP impurity generating a slurry;
(b) washing said slurry containing at least one HCP impurity to remove unbound material;
(c) eluting the bound HCPs to produce an enriched HCP sample;
(d) subjecting said enriched HCPs to enzymatic digestion conditions to produce a peptide digest; and
(e) subjecting said peptide digest to mass spectrometry/mass spectrometry (MS/MS) analysis to enrich, identify and/or characterize said at least one HCP impurity.
Embodiment 2. The method of embodiment 1, wherein said enriched HCPs are subjected to a denaturant to produce a denatured sample.
Embodiment 3. The method of embodiment 2, wherein said denaturant comprises heat, high pH, low pH, reducing agents, or chaotropic agents.
Embodiment 4. The method of any one of embodiments 1-3, wherein said enriched HCPs are subjected to an alkylating agent to produce an alkylated sample.
Embodiment 5. The method of embodiment 4, wherein said alkylating agent comprises iodoacetamide (IOA/IAA), chloroacetamide (CAA), acrylamide (AA), N-ethylmaleimide (NEM), methyl methanethiosulfonate (MMTS), 4-vinylpyridine, or combinations thereof.
Embodiment 6. The method of any one of embodiments 1-5, wherein said enriched HCPs are subjected to a reducing agent to produce a reduced sample.
Embodiment 7. The method of embodiment 6, wherein said reducing agent comprises dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof.
Embodiment 8. The method of any one of embodiments 1-7, wherein said enriched HCPs are subjected to a denaturant, an alkylating agent, and a reducing agent to produce a denatured, reduced, and alkylated sample.
Embodiment 9. The method of any one of embodiments 1-8, wherein the viral vector is an AAV vector.
Embodiment 10. The method of embodiment 9, wherein said AAV vector comprises a serotype selected from AAV1, AAV2, AAV3, AAV4, AAV5, AAV6, AAV7, AAV8, AAVrh8, AAV2/8, AAV9, AAV10, AAV11, AAV12, variations thereof and combinations thereof.
Embodiment 11. The method of any one of embodiments 1-10, wherein the amount of sample containing at least one host cell protein impurity is about 20 μL to about 100 μL.
Embodiment 12. The method of any one of embodiments 1-11, wherein the library of peptide ligands comprises a binding solution with a pH of about pH 6.0 to about pH 8.0, optionally wherein said pH is about 7.0.
Embodiment 13. The method of any one of embodiments 1-12, wherein the volume of the library of peptide ligands is about 0.4 μL to about 15 μL.
Embodiment 14. The method of any one of embodiments 1-13, wherein said enzymatic digestion conditions comprise contacting said denatured, reduced and alkylated sample with at least one digestive enzyme.
Embodiment 15. The method of embodiment 14, wherein said at least one digestive enzyme comprises trypsin.
Embodiment 16. The method of any one of embodiments 1-15, wherein said mass spectrometer is an electrospray ionization mass spectrometer, nano-electrospray ionization mass spectrometer, or a triple quadrupole mass spectrometer.
Embodiment 17. The method of any one of embodiments 1-16, wherein said mass spectrometer is coupled to a liquid chromatography system.
Embodiment 18. A method of enriching, identifying and/or characterizing at least one host cell protein (HCP) impurity in a sample containing at least one viral vector, comprising:
(a) contacting a sample including at least one HCP impurity to a solid support, wherein said solid support has attached thereto a library of peptide ligands capable of interacting with said at least one HCP impurity generating a slurry;
(b) washing said slurry containing at least one HCP impurity to remove unbound material;
(c) eluting bound HCPs to produce an enriched HCP sample;
(d) subjecting said enriched HCP sample to a denaturant to produce a denatured sample;
(e) subjecting said denatured sample to a reducing agent to produce a denatured and reduced sample;
(f) subjecting said denatured and reduced sample to an alkylating agent to produce a denatured, reduced and alkylated sample;
(g) subjecting said denatured, reduced and alkylated sample to enzymatic digestion conditions to produce a peptide digest; and
(h) subjecting said peptide digest to liquid chromatography-mass spectrometry/mass spectrometry (LC-MS/MS) analysis to identify and/or characterize said at least one HCP impurity.
Embodiment 19. The method of embodiment 18, wherein the viral vector is an AAV vector.
Embodiment 20. The method of embodiment 19, wherein said AAV vector comprises a serotype selected from AAV1, AAV2, AAV3, AAV4, AAV5, AAV6, AAV7, AAV8, AAVrh8, AAV2/8, AAV9, AAV10, AAV11, AAV12, variations thereof and combinations thereof.
Embodiment 21. The method of embodiment 20, wherein the amount of sample containing at least one host cell protein impurity is about 20 μL to about 100 μL.
Embodiment 22. The method of any one of embodiments 18-21, wherein said volume of interacting peptide ligands is about 0.4 μL to about 15 μL.
Embodiment 23. The method of any one of embodiments 18-22, wherein the library of peptide ligands comprises a binding solution with a pH of about pH 6.0 to about pH 8.0, optionally wherein said pH is about 7.0.
Embodiment 24. The method of any one of embodiments 18-23, wherein said denaturant comprises heat, high pH, low pH, reducing agents, or chaotropic agents.
Embodiment 25. The method of any one of embodiments 18-24, wherein said alkylating agent comprises iodoacetamide (IOA/IAA), chloroacetamide (CAA), acrylamide (AA), N-ethylmaleimide (NEM), methyl methanethiosulfonate (MMTS), 4-vinylpyridine, or combinations thereof.
Embodiment 26. The method of any one of embodiments 18-25, wherein said reducing agent comprises dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof.
Embodiment 27. The method of any one of embodiments 18-26, wherein said enzymatic digestion conditions comprise contacting said denatured, reduced, and alkylated sample to at least one digestive enzyme, optionally wherein said at least one digestive enzyme comprises trypsin.
Embodiment 28. The method of any one of embodiments 18-27, wherein said mass spectrometry system is electrospray ionization mass spectrometer, nano-electrospray ionization mass spectrometer, or an Orbitrap-based mass spectrometer.
Embodiment 29. The method of any one of embodiments 18-28, wherein said mass spectrometer is coupled to said liquid chromatography system.

### EXAMPLES

The following examples are provided to further describe some of the embodiments disclosed herein. The examples are intended to illustrate, not to limit, the disclosed embodiments.

### Materials

An exemplary solid support with a library of peptide ligands attached thereto are ProteoMiner™ beads. ProteoMiner™ protein enrichment small-capacity kit was obtained from Bio-Rad (Hercules, CA). Chromatography solvents were of LC-MS grade from Fisher Scientific (Waltham, MA). AAV reference materials including AAV1 empty capsids, AAV5 empty capsids, AAV8 empty capsids, AAV9 empty capsids and AAV8 full capsids were obtained from Charles River. Sodium deoxycholate (SDC), sodium lauroyl sarcosinate (SLS), 2-Chloroacetamide (CAA), Tris(2-carboxyethyl)phosphine hydrochloride (TCEP), Proteomics Dynamic Range Standard Set UPS2 were obtained from Sigma-Aldrich (St. Louis, MO). lodoacetamide (IAM), Dithiothreitol (DTT), Trifluoracetic acid (TFA), UltraPure 1 M Tris-HCl pH 8.0 were obtained from Thermo Fisher. AAV8 and AAV9 empty capsids with spiked in UPS2 was prepared by dilution of UPS2 (10.6 μg/ampule protein) in 32 μL water and the addition of 2 μL diluted UPS2 into 500 μL AAV reference material with empty capsids.

### Sample Preparation Using ProteoMiner™ Protein Enrichment Kit

ProteoMiner™ Protein Enrichment kit was used to enrich proteins in samples. HCPs from AAV reference materials were enriched using ProteoMiner™ enrichment beads following a similar protocol as described by Chen et al. with some modifications. The AAV reference materials were stored in PBS buffer containing 0.01% Pluronic F-68 (pH 7). The viral particle titer of the AAV reference materials with empty capsids ranged from 1.20×10¹² VP/mL to 2.30×10¹² VP/mL, and the viral genome titer of AAV8 reference material with full capsids was 7.97×10¹¹ GC/mL. The pH of the AAV reference material empty capsids were adjusted by adding 50 mM acetic acid (pH 6) or 50 mM Tris-HCl, pH 8.0 (pH 8). ProteoMiner™ beads were washed three times with 200 μL of wash buffer provided in the enrichment kit and followed by one water wash. The beads were resuspended in water and 10 μL of beads slurry was added to AAV reference material and incubated by rotation at room temperature for 2.5 hours. The slurry beads with enriched proteins were loaded onto an in-house made tip, centrifuged at 200×g for 5 minutes until dry. Subsequently, the beads were washed three times by adding 100 μL of wash buffer, followed by one water wash. Finally, the enriched proteins were eluted three times by addition of 10 μL of PTS buffer containing 12 mM SDC, 12 mM SLS, 10 mM TCEP and 40 mM CAA. The eluent was then denatured, reduced and alkylated in PTS buffer at 95° C. for 5 minutes, cooled down to room temperature and subsequently diluted by adding 120 μL of 0.1 M Tris-HCl, pH 8.0 and digested by 10 ng trypsin overnight at 37° C. The digested peptides were acidified by adding 10 μL of 10% TFA and centrifuged at 14,000×g for 20 minutes, and peptide-containing supernatant was collected for sequential desalting and nano LC-MS/MS analysis.

### Sample Preparation with Direct Digestion

Direct digestion was performed by adding 10 μL of denaturing buffer containing 36 mM SDC/SLS, 30 mM TCEP, 120 mM CAA into 20 μL of AAV reference material and heated at 95° C. for 5 minutes. The denatured, reduced and alkylated AAV reference material was then diluted by adding 120 μL of 0.1 M Tris-HCl, pH 8.0 and digested with 100 ng trypsin overnight at 37° C. The digested peptides were acidified by adding 10 μL of 10% TFA and centrifuged at 14,000×g for 20 minutes, and the peptide-containing supernatant was collected for sequential desalting and nano LC-MS/MS analysis.

### Nano LC-MS/MS Analysis

The peptide mixture was resuspended in 12 μL 0.1% formic acid (FA) in water and 10 μL was analyzed using an UltiMate 3000 RSLC nano LC system interfaced to an Orbitrap Exploris 480 mass spectrometer (Thermo Fisher Scientific). Peptides were loaded onto a trap column (20 cm×0.075 mm Acclaim PepMap 100 C18) at 5 μL/min and analytical column (30 cm×0.075 mm, 1.7 μm, 100 Å, CoAnn Technologies) at 0.25 μL/min sequentially for desalting and separation. The mobile phase A was 0.1% FA in water and mobile phase B was 0.1% FA in acetonitrile. The gradient was set as follows: 4% mobile phase B for 6 min, up to 20% B during 84 min time period, an increase to 36% B within 35 min, and a steep increase to 95% B within 9 min, and then held for 9 min at 95% B. The Orbitrap Exploris was operated in a data-dependent mode (DDA), with full MS scan at 60,000 resolution at m/z 380-1600 and a predicted automatic gain control (AGC) value of 3e⁶ with maximal injection time of 20 ms, and MS/MS event at a resolution of 15,000 resolutions, predicted AGC of 1e⁵ with maximal injection time of 60 ms at m/z 200-2000.

### Data Analysis

The mass spectrometry raw data was searched against the UniProt Homo Sapiens (version 2022) with Proteome Discoverer software (version 2.4) using Sequest HT and Mascot search engines. The spectra were searched with 10 ppm mass tolerance for precursor, and 0.02 Da for fragment, for tryptic digestion with a maximum of two missed cleavage sites. Carbamidomethylation of cysteines (+57.0214 Da) was included as static modification and oxidation (+15.9949 Da) on methionine and deamidation (+0.984 Da) on asparagine were included as variable modification. The false-discovery rates of proteins and peptides were set at 0.01 and the HCPs were positively identified when at least two unique peptides were found. Example 1. Enrichment Method Optimization using ProteoMiner™ Protein Enrichment Kit

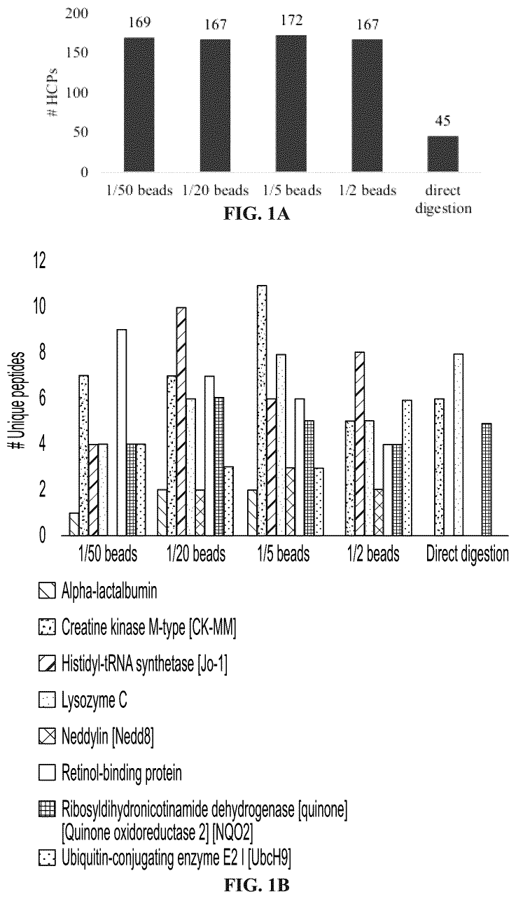

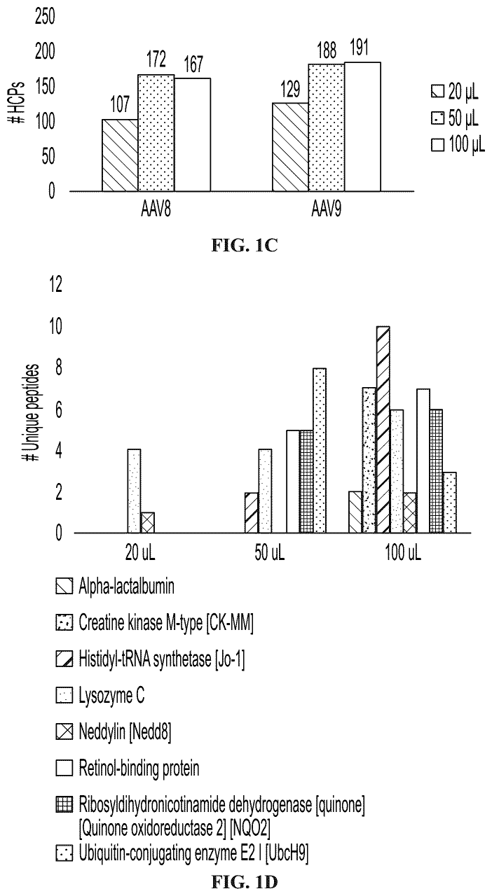

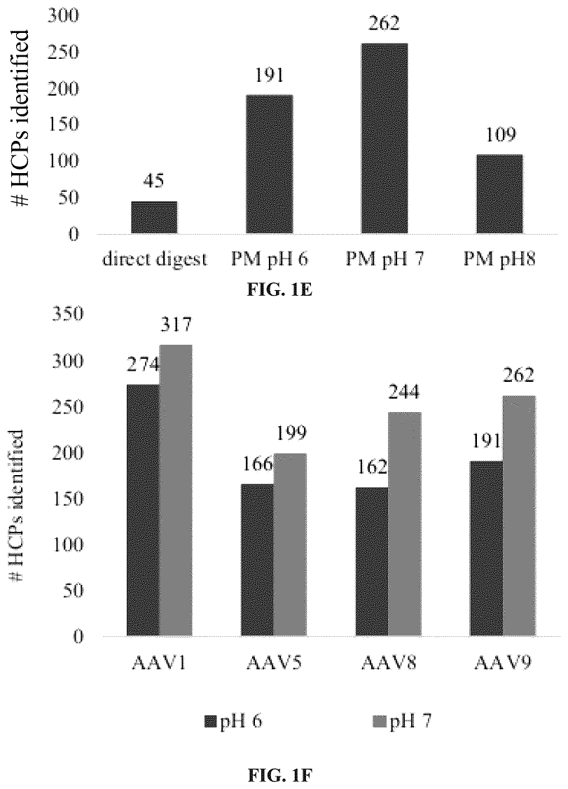

The effectiveness of enriching HCPs using different ProteoMiner™ beads amount was evaluated with UPS2 spiked into pH adjusted AAV8 reference material with empty capsids. ProteoMiner™ is an exemplary library of peptide ligands. UPS2 is a commercially available proteomics standard comprising 48 human proteins at a wide dynamic range of concentrations spanning many orders of magnitude. The pH of the AAV8 reference material was adjusted to pH 6.0 by the addition of 2.5 μL of 1M acetic acid and then added into 500 μL according to previous ProteoMiner™ enrichment protocol. 1/16 UPS2 (0.66 μg protein) was spiked into 500 μL pH adjusted AAV8 empty capsids to evaluate the detection limit. The method was conducted with varying amounts of ProteoMiner™ beads including ½ of ProteoMiner™ kit (10 μL beads), ⅕ ProteoMiner™ kit (4 μL beads), 1/20 ProteoMiner™ kit (1 μL beads) and 1/50 ProteoMiner™ kit (0.4 μL beads). The varying amounts of beads were incubated with 100 μL of UPS2 spiked-in, pH adjusted AAV8 material and the number of HCPs identified were compared with an alternative direct digestion method. An average of 167 HCPs were detected after ProteoMiner™ enrichment while only 45 HCPs were detected with direct digestion as shown in FIG. 1A. All UPS2 standards above 6.25 fmol were detected after applying varying amounts of enrichment beads as shown in Table 1. In comparison, Table 1 also shows not all UPS2 standards with a concentration less than 4 ng/mL were detectable using the direct digestion method. There were no discernible variations in the number of HCPs identified (FIG. 1A) or the number of unique peptides identified from UPS2 standards at 6.25 fmol (FIG. 1B) when varying amounts of beads were applied. This suggests the enrichment efficiency, with respect to detection limit and sensitivity, remains unaffected by the quantity of beads used. Therefore, 1 μL of enrichment beads was used for all subsequent experiments to minimize the variation of dispensed beads amount and to conserve the experimental material.

TABLE 1
The detection limit of HCPs in UPS2 standards from a UPS2-spiked in empty
capsid AAV8 sample comparing an enrichment method of the present disclosure
with varying concentrations of ProteoMiner ™ beads and direct digestion.

| | UPS2 Amount | | | | | | | | |
|---|---|---|---|---|---|---|---|---|---|
| | (fmol) in | | UPS2 | | | | | | direct |
| UniProt Protein Name | 100 uL AAV8 | MW | amount | UPS2 | 1/50 | 1/20 | ⅕ | ½ | digestion |
| [Synonym] | empty capsids | (Da) | (ng) | (ng/mL) | Unique peptides | | | | |
| Carbonic anhydrase 1 | 625 | 28738 | 17.96 | 179.61 | 17 | 16 | 15 | 15 | 12 |
| Carbonic anhydrase 2 | | 29115 | 18.20 | 181.97 | 18 | 16 | 17 | 21 | 15 |
| Complement C5 | | 8536 | 5.34 | 53.35 | 11 | 10 | 16 | 16 | 11 |
| [Complement C5a] | | | | | | | | | |
| Hemoglobin alpha chain | | 15126 | 9.45 | 94.54 | 9 | 9 | 9 | 10 | 7 |
| Hemoglobin beta chain | | 15867 | 9.92 | 99.17 | 12 | 11 | 11 | 12 | 7 |
| Leptin | | 16158 | 10.10 | 100.99 | 6 | 6 | 6 | 7 | 2 |
| Serum Albumin | | 66357 | 41.47 | 414.73 | 79 | 76 | 80 | 90 | 52 |
| Ubiquitin | | 10597 | 6.62 | 66.23 | 9 | 8 | 10 | 7 | 7 |
| Catalase | 62.5 | 59625 | 3.73 | 37.27 | 21 | 19 | 21 | 21 | 22 |
| Cytochrome b5 | | 16022 | 1.00 | 10.01 | 7 | 7 | 5 | 6 | 4 |
| Epidermal Growth Factor | | 6353 | 0.40 | 3.97 | 3 | 3 | 3 | 2 | |
| Myoglobin C | | 17053 | 1.07 | 10.66 | 7 | 11 | 10 | 12 | 7 |
| NAD(P)H dehydrogenase | | 30736 | 1.92 | 19.21 | 7 | 6 | 8 | 8 | 6 |
| [quinone] 1 [DT Diaphorase] C | | | | | | | | | |
| Peptidyl-prolyl cis-trans | | 20176 | 1.26 | 12.61 | 9 | 11 | 10 | 12 | 6 |
| isomerase A [Cyclophilin A] | | | | | | | | | |
| Peroxiredoxin 1 | | 21979 | 1.37 | 13.74 | 16 | 13 | 13 | 14 | 10 |
| Small ubiquitin-related | | 38815 | 2.43 | 24.26 | 8 | 14 | 12 | 15 | 10 |
| modifier 1 [SUMO-1] | | | | | | | | | |
| Alpha-lactalbumin | 6.25 | 14078 | 0.09 | 0.88 | 1 | 2 | 2 | 1 | |
| Creatine kinase M-type | | 43101 | 0.27 | 2.69 | 7 | 7 | 11 | 5 | 6 |
| [CK-MM] | | | | | | | | | |
| Histidyl-tRNA synthetase | | 58233 | 0.36 | 3.64 | 4 | 10 | 6 | 8 | |
| [Jo-1] | | | | | | | | | |
| Lysozyme C | | 14701 | 0.09 | 0.92 | 4 | 6 | 8 | 5 | 8 |
| Neddylin [Nedd8] | | 9072 | 0.06 | 0.57 | 1 | 2 | 3 | 2 | |
| Retinol-binding protein | | 21071 | 0.13 | 1.32 | 9 | 7 | 6 | 4 | |
| Ribosyldihydronicotinamide | | 25821 | 0.16 | 1.61 | 4 | 6 | 5 | 4 | 5 |
| dehydrogenase [quinone] | | | | | | | | | |
| [Quinone oxidoreductase 2] | | | | | | | | | |
| [NQO2] | | | | | | | | | |
| Ubiquitin-conjugating | | 18007 | 0.11 | 1.13 | 4 | 3 | 3 | 6 | |
| enzyme E2 I [UbcH9] | | | | | | | | | |

### Example 2. Parameter Optimization Using ProteoMiner™ Protein Enrichment Kit

The method of the present disclosure was further optimized by comparing a range of sample volumes. Due to the low titer during drug production, the quantity of AAV materials was often restricted. The method was conducted with AAV8 reference material with UPS2 spiked in and AAV9 reference material adjusted to pH 6.0 for HCP analysis. Varying volumes of reference material, 20 μL, 50 μL, and 100 μL, were compared to determine the minimum sample amount needed to achieve detection for the analysis of HCPs. As shown in FIG. 1C, the number of HCPs identified was the consistent for both the 50 μL and 100 μL sample volumes. An increase of 60% and 46% number of HCPs were observed in AAV8 and AAV9 reference material when 50 μL or 100 μL of sample was used compared to 20 μL, respectively. However, only 5 out of 8 UPS2 at 6.25 fmol were detected from 50 μL sample compared to 8 out 8 UPS2 at 6.25 fmol were detected from 100 μL sample, as shown in FIG. 1D.

Furthermore, the identities of HCPs enriched from 50 μL AAV reference materials were found approximately 60% overlapped with HCPs enriched from 100 μL as shown in FIG. 1D. The results indicate that a detection limit of 0.5 ng/mL can be achieved when 100 μL of sample was applied, while the detection limit increased with the amount of sample used for enrichment decreased. 100 μL of AAV reference material was used for the rest of the experimental procedure.

The binding efficiency of enrichment beads on antibody drug substances was reported to be affected differently by pH. Similarly, the binding efficiency of enrichment beads on HCPs in AAV products might vary from that in antibody drug substances. Therefore, the method of the present disclosure was further optimized to evaluate the pH of the binding solution for AAV products. The AAV reference materials were originally stored at PBS buffer containing 0.01% Pluronic F-68 (pH 7.0). The pH was adjusted to 6.0 by adding 50 mM acetic acid and adjusted to 8 by adding 50 mM Tris-HCl, pH 8.0. The enrichment was performed by incubating 1 μL of ProteoMiner™ beads with 100 μL of AAV reference material. For AAV9 reference material with empty capsids, 191 HCPs were detected in acidic condition while 262 HCPs were detected in the neutral condition and 109 HCPs were detected in basic condition as shown in FIG. 1E.

To evaluate whether a neutral pH is the optimal incubation condition for all AAV serotypes for HCP enrichment, the method of the present disclosure was used to compare AAV1, AAV5 and AAV8 reference materials with empty capsids at pH 6.0 and pH 7.0. As shown in FIG. 1F, the number of HCPs detected from different AAV serotypes increased 15.6% to 50.6% when AAV reference materials were incubated with enrichment beads at neutral pH conditions as compared to acidic pH conditions. Therefore, neutral pH was chosen for enriching HCPs from AAV samples.

The optimized conditions were used to evaluate the reproducibility of the method of the present disclosure. As shown in FIG. 2A, approximately 146 HCPs were identified with the enrichment method of the present disclosure from AAV8 reference material with full capsids, which is 4.3-fold higher than the number of HCPs identified using direct digestion. Over three preparations, 131 common HCPs were identified, equivalent to 80.9% of all proteins as shown in FIG. 2B. The high reproducibility suggests high confidence in protein identification, which is critical for HCP analysis.

### Example 3. Case Studies Using ProteoMiner™ Protein Enrichment Kit

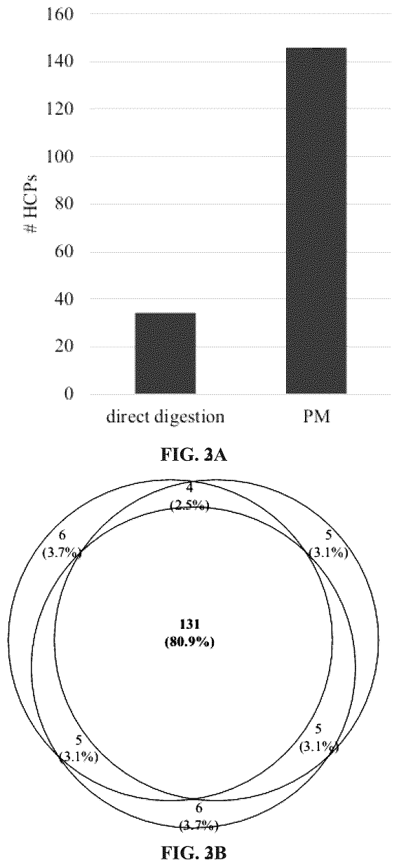

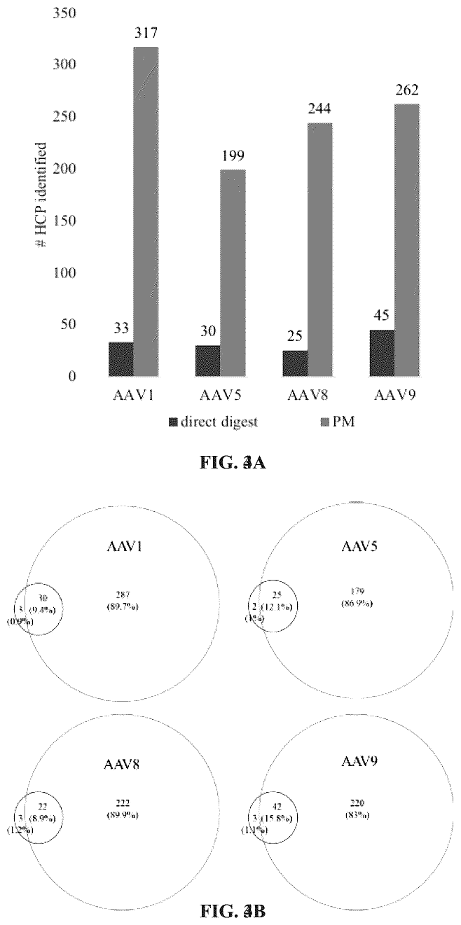

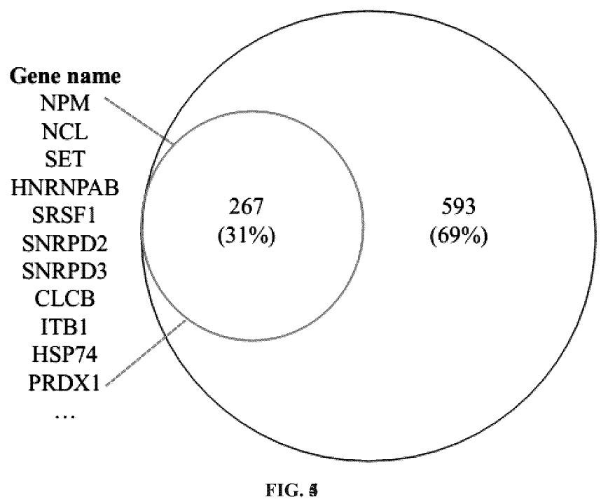

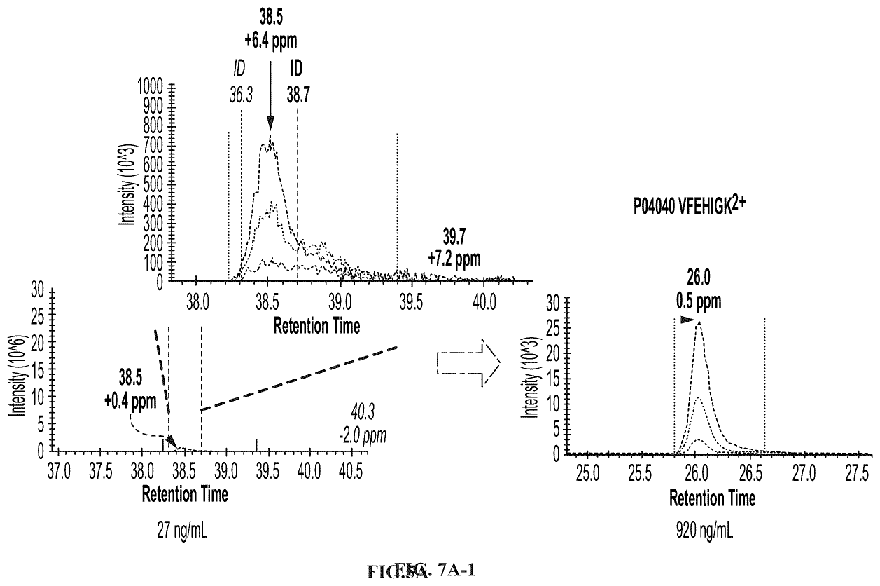

The optimized method described in Example 2 was used with several different AAV serotypes and compared to the direct digestion method. A significant increase in the number of identified HCPs from AAV1, AAV5, AAV8, and AAV9 reference materials with empty capsids was observed, as shown in FIG. 3A. This enhancement was observed across all AAV serotypes. FIG. 3A illustrates over 80% of the newly identified HCPs were detected using the enrichment method of the present disclosure, revealing less than 1.2% of missing proteins compared to the direct digestion approach. These findings indicate that the method of the present disclosure is comprehensive and capable of detecting a large number of HCPs present in low abundance, which are otherwise not detectable through direct digestion.

In total, 860 HCPs were identified from AAV1, AAV5, AAV8 and AAV9 reference material by ProteoMiner™ enrichment coupled with nano liquid chromatography-mass spectrometry/mass spectrometry (LC-MS/MS). FIG. 4 illustrates the results of the analysis, where out of the 860 HCPs identified, 267 have been previously reported in literature from AAV products produced by the human cell line HEK293. These proteins include frequently reported HCPs such as nucleophosmin, protein SET, nucleolin, as well as RNA binding proteins like small nuclear ribonucleoproteins Sm D2, D3, heterogeneous nuclear ribonucleoprotein A/B, and PABP. Additionally, some HCPs commonly found in monoclonal antibodies (mAbs), such as peroxiredoxin 1, ubiquitin, actin, and Hsc70, were also detected in AAV products, potentially regulating cell functions. Moreover, the enrichment method of the present disclosure identified 593 HCPs that had never been reported previously.

The optimized method was used to evaluate the dynamic range reduction in AAV9 reference material. AAV9 empty capsid reference material was spiked with 0.264 μg of UPS2 protein and range of protein detection was determined. Seven UPS2 standards ranging between 0.1 ng/mL and 414.7 ng/mL were chosen for comparison. The concentration of each spiked-in UPS2 standard was calculated by comparing the relative abundance of UPS2 standard peptides versus capsid protein peptides for samples prepared by the present optimized enrichment method and direct digestion method. The enrichment factor was calculated by comparing the calculated concentration with spiked-in concentration as shown in Table 2. The enrichment factor of UPS2 standards prepared by direct digestion were close to 1 since no enrichment occurred, as shown in FIG. 4. Some standards, such as serum albumin and peroxiredoxin, were slightly larger than 1 for the direct digestion, and this is likely due to pre-existing HCPs in the AAV reference material. The enrichment factor for ProteoMiner™ treated samples ranged from 1.5 to 25, as shown in FIG. 4.

TABLE 2
Enrichment factor of UPS2 standards detected from UPS2-spiked in AAV9 empty
capsids using direct digestion and ProteoMiner ™ enrichment.

| | | | Direct | PM | Direct | PM | PM/ |
|---|---|---|---|---|---|---|---|
| | | Spiked in | digestion | enrichment | digestion | enrichment | direct |
| | accession | concentration | Enrichment | Enrichment | Dynamic | Dynamic | digestion |
| HCP | # | (ng/mL) | factor | factor | range | range | ratio |
| serum | P02768 | 414.7 | 1.35 | 13.53 | 2.96E−02 | 2.96E−01 | 10.00 |
| albumin | | | | | | | |
| hemoglobin | P68871 | 99.2 | 1.13 | 7.31 | 2.48E−02 | 1.60E−01 | 6.44 |
| beta chain | | | | | | | |
| catalase | P04040 | 37.3 | 0.72 | 24.66 | 1.58E−03 | 5.40E−02 | 34.14 |
| peroxiredoxin | Q06830 | 13.7 | 1.80 | 6.50 | 3.92E−03 | 1.42E−02 | 3.63 |
| NQO2 | P16083 | 1.6 | 1.17 | 6.46 | 2.54E−04 | 1.40E−03 | 5.51 |
| neddylin | Q15843 | 0.6 | — | 1.49 | — | 3.46E−04 | |
| [Nedd8] | | | | | | | |
| thioredoxin | P10599 | 0.1 | — | 25.57 | — | 7.20E−04 | |

1. Enrichment factor was calculated by equation 1:

$$Enrichment factor = \frac{\begin{matrix}average peak area of three highest abundant peptides from \\ spiked - in HCPs \times Mw of HCP\end{matrix}}{\begin{matrix}concentration of HCP \times average peak area of three highest \\ abundant peptides from capsid protein \times Mw of capsid protein\end{matrix}}$$

2. Direct digestion dynamic range was calculated by equation 2:

$$Dynamic range = \frac{\begin{matrix}average peak area of three highest abundant peptides from \\ spiked - in HCPs\end{matrix}}{\begin{matrix}average peak area of three highest abundant peptides from \\ capsid protein\end{matrix}}$$

3. PM/direct digestion ratio was calculated by dividing PM enrichment factor by direct digestion enrichment factor

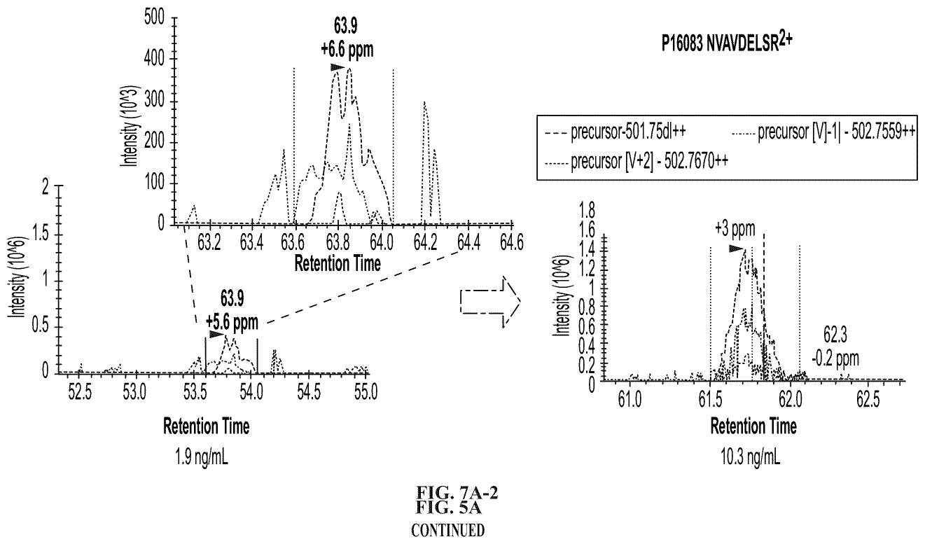

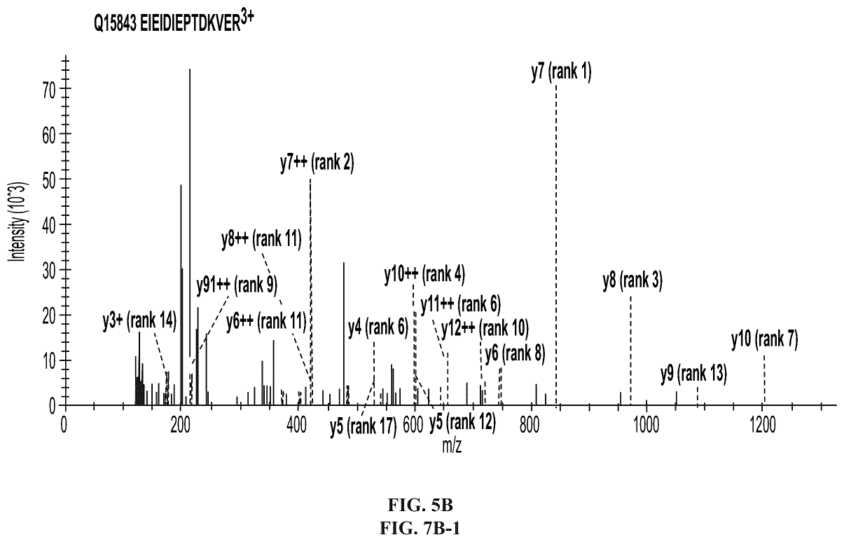

For UPS2 with a spiked-in concentration above 1.6 ng/mL, the dynamic range between UPS2 peptide and AAV capsid protein peptide was ranging between 1.4E⁻⁰³ and 3.0E⁻⁰¹ for enrichment method and 2.5E⁻⁰⁴ and 3.0E⁻⁰² for direct digestion, which demonstrated a 3.6-fold to 34.1-fold increase after enrichment. FIG. 5A demonstrates the signal changes of catalase peptide VFEHIGK²⁺ (SEQ ID NO: 1) and Ribosyldihydronicotinamide dehydrogenase [quinone] [Quinone oxidoreductase 2] [NQO2] peptide NVAVDELSR²⁺ (SEQ ID NO: 2). For neddylin at 0.6 ng/mL and thioredoxin at 0.1 ng/mL, the dynamic range between UPS2 peptide and AAV capsid protein peptides was 3.5E⁻⁰⁴ and 7.2E⁻⁰⁴ after enrichment, and it can be predicted that the dynamic range between these two UPS2 peptides and capsid protein peptides would be in the range of E⁻⁰⁵ with direct digestion. Because of the potential low range of neddyline and thioredoxin, and most likely other host cell proteins in low ng/ml or below 1 ng/ml, they were not detectable with direct digestion preparation. FIG. 5B shows the MS2 spectrum of neddylin peptide EIEIDIEPTDKVER³⁺ (SEQ ID NO: 3) at 0.6 ng/mL and thioredoxin peptide TAFQEALDAAGDK²⁺ (SEQ ID NO: 4) at 0.1 ng/mL after enrichment. A significant improvement of sensitivity in detecting these low abundance HCPs (<1 ng/mL) was observed by applying the present enrichment method.

### Example 4. Complete Detergent Removal after Enrichment Beads Treatment

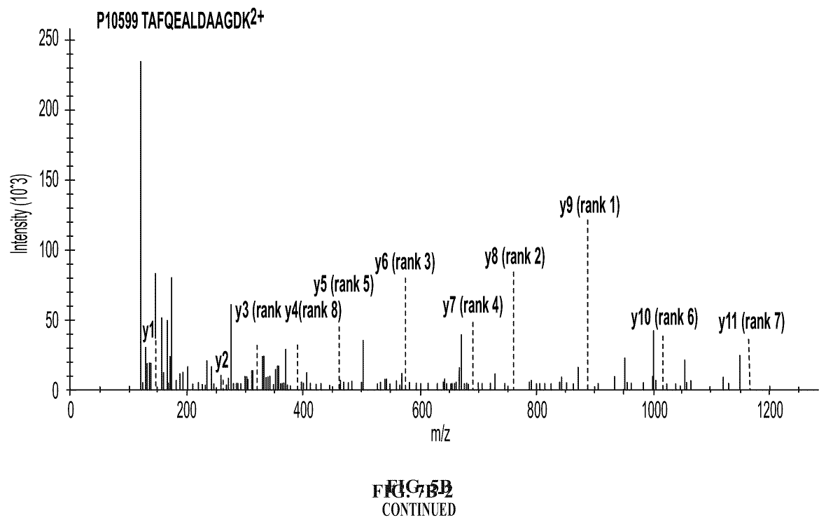

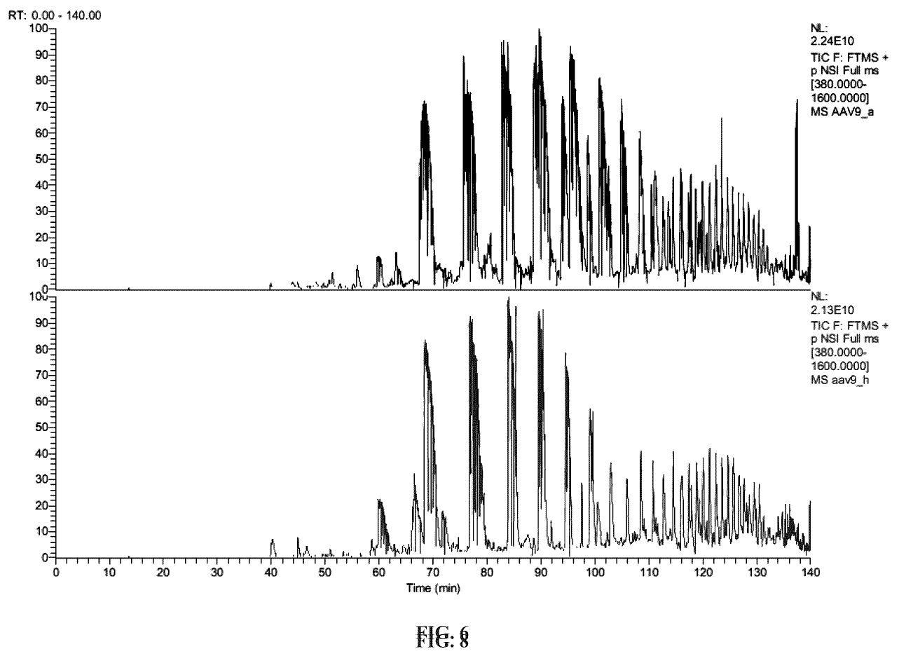

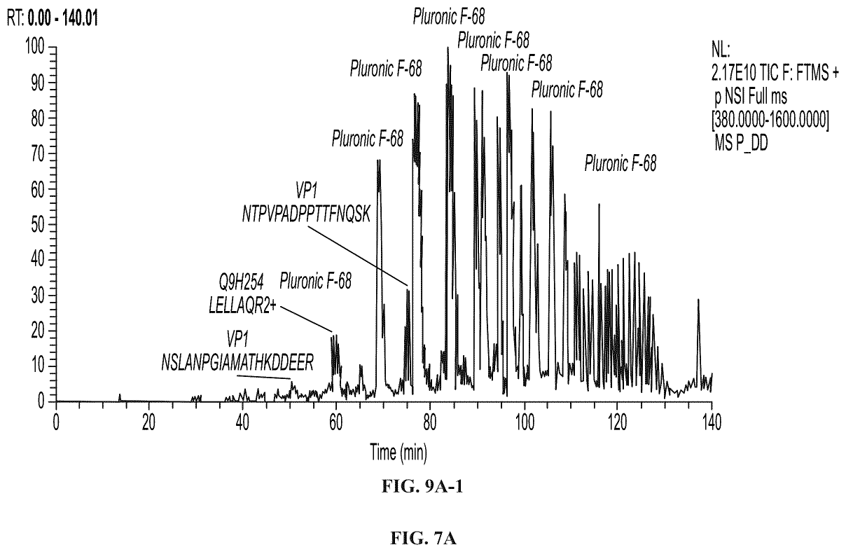

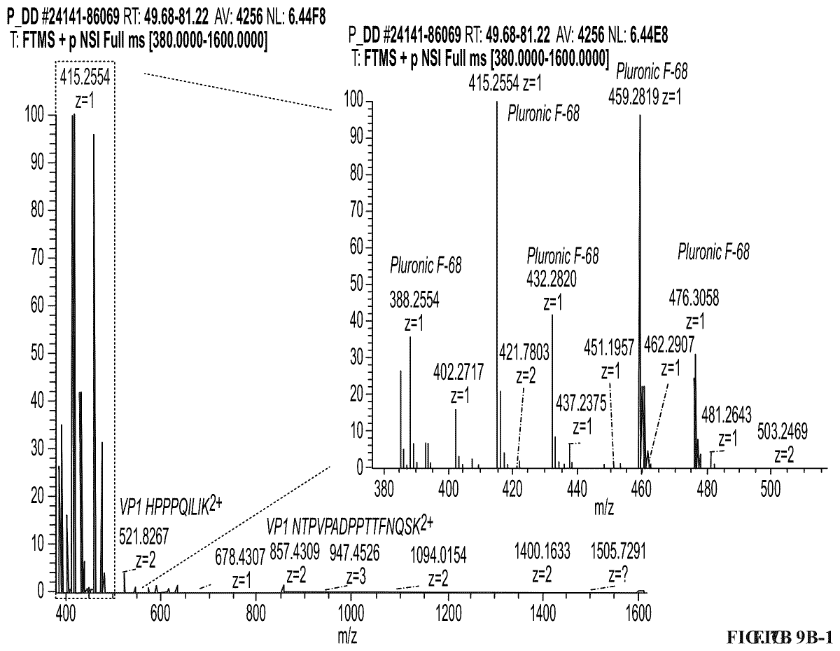

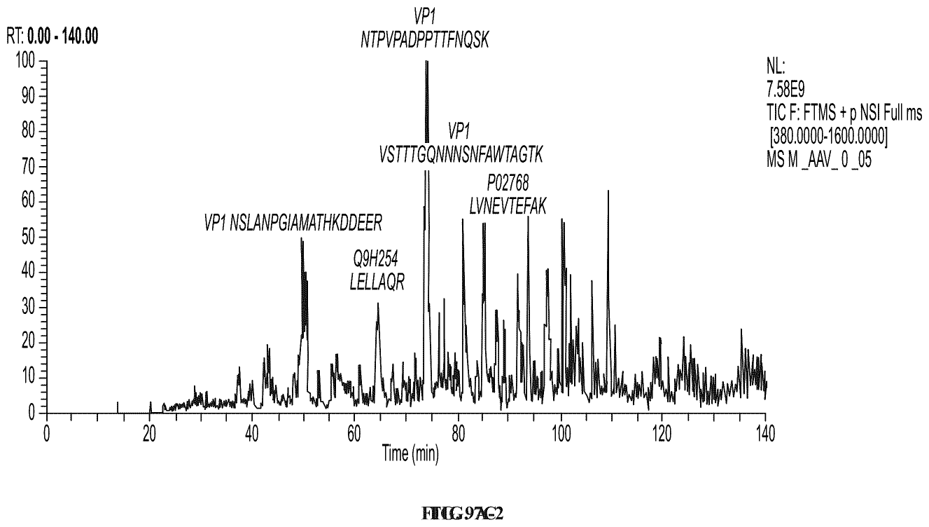

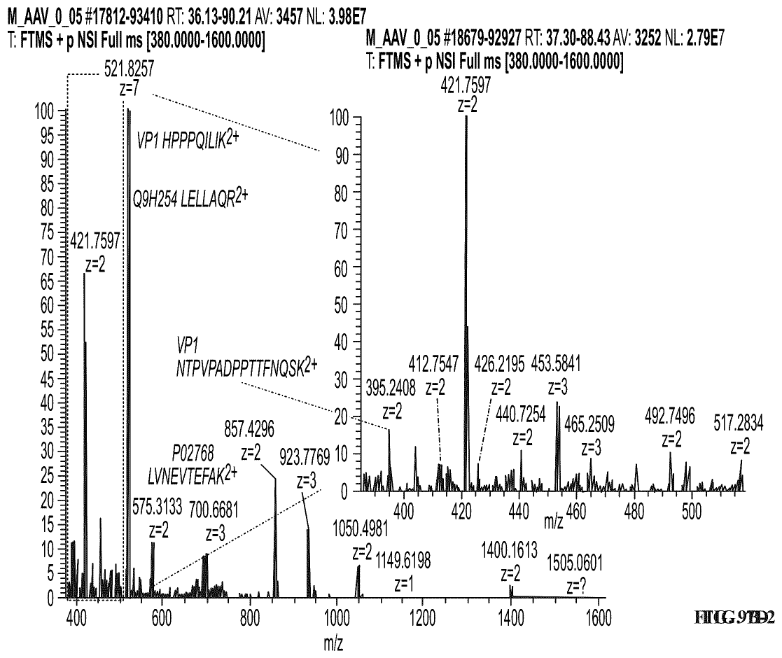

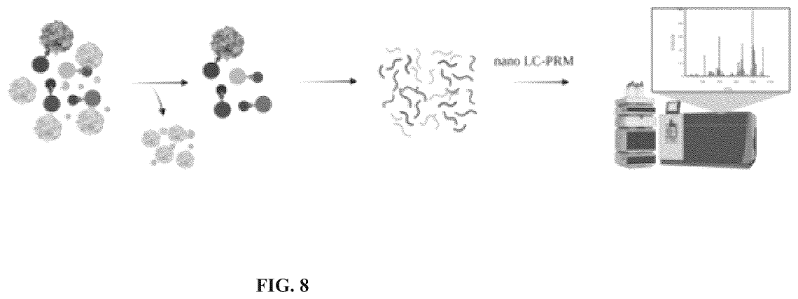

ProteoMiner™ protein enrichment technology is utilized to enrich for low abundant proteins, therefore, only proteins, such as antibodies, AAV capsid proteins and HCPs bind to the beads. Detergents, such as polysorbate and Pluronic F-68, which are commonly used in mAb drug products and/or AAV products, do not bind to the enrichment beads and thus will be removed from the protein mixtures after enrichment. FIG. 6 shows the total ion chromatograms (TIC) comparing direct digestion (top panel) to an AAV product prepared using on-filter direct digestion (bottom panel). The molecular weight cut off filter encountered difficulty in removing these detergents, thus filtration is not useful in reducing interference from the detergent. In addition, a significant loss of HCPs during buffer exchange was found due to their adhesion to the filter membrane. In comparison, FIGS. 7A and 7C show the TIC and corresponding MS signals of the direct digestion method where the Pluronic F-68 were over 20 times higher than those of the viral protein peptides. This significant difference in signal levels posed a major challenge for the detection of HCP peptides since the detectable HCP peptides were 100 to 10,000 times lower in abundance compared to viral protein peptides. FIG. 7B and FIG. 7D show the TIC and corresponding MS spectrums of the peptide mixtures after the enrichment method of the present disclosure. The detergent peaks were entirely eliminated by the enrichment beads, and certain HCP peptides were observed at similar levels to viral protein peptides in the TIC profile.

### Example 5. Analysis of Host Cell Proteins in AAV Products with ProteoMiner™ Protein Enrichment Technology

Information regarding HCPs in gene therapy products is limited and has not been integrated into host cell engineering or purification processes. In this study, ProteoMiner™ beads effectively enrich HCPs in adeno-associated virus (AAV) products and simultaneously remove the detergent Pluronic F-68 without loss of low-abundance HCPs. There was up to 34-fold increase in the enrichment of HCPs compared to direct digestion. The detection limit was significantly lowered, with the ability to detect HCPs at levels as low as 0.1 ng/ml after ProteoMiner™ treatment. The findings from this study provide insights into HCPs in AAV products and may facilitate process development and host cell line optimization. The high sensitivity of this approach also facilitates detection of low-abundance HCPs, thereby contributing to the safety and quality of AAV-based gene therapy product.

### Introduction

Host cell proteins (HCPs) are proteins that are produced by the host organism and remain in drug products (DPs). The concentration of HCPs is usually low in DPs after the multiple stringent purification steps applied during processing. However, HCPs are considered a significant quality attribute despite their low abundance, because of their potential risk of including immunogenicity, protein clipping or glycosylation, degradation, and excipient destabilization.

A major challenge in HCP analysis in gene therapy is the limited product quality. HCP enrichment methods for monoclonal antibody (mAb) drug preparations usually require at least 1 mg of mAb to accumulate sufficient HCPs for MS detection. However, the quantity of gene therapy products, such as AAV material available for HCP analysis, is usually limited to 10 ug or less. Thus, approaches such as the molecular weight cut-off method cannot be applied to separate HCPs from AAV capsid proteins because most of the low-abundance HCPs would be adsorbed by the filter, and little would flow through the membrane. The detergent Pluronic F-68, which is commonly used in AAV products, is also difficult to remove by filtration, and the MS signals of the detergent severely interfere with the detection of HCP peptides. Anti-HCP polyclonal antibodies used for immunocapture of HCPs from mAb drugs have been found to be insufficient to recognize all HCPs from AAV products. In addition, because the capsid protein does not contain disulfide bonds, limiting the amount of trypsin used cannot prevent capsid protein digestion; therefore, HCP analysis in AAV material cannot be improved by limited digestion.

ProteoMiner™ beads are a library of bead-based peptide ligands that bind diverse protein types. In this study, an HCP analysis method for AAV products by using ProteoMiner™ enrichment beads was developed. The approach improved the dynamic range of HCP detection by one to two orders of magnitude with respect to direct digestion, and achieved a 5-fold to 10-fold increase in the number of HCPs detected from AAV material. The detergent was completely removed, thus avoiding interference in the detection of HCP peptides. This approach could potentially be applied to other gene therapy products, and used to troubleshoot and identify potentially problematic HCPs for risk assessment.

### Materials and Methods

A ProteoMiner™ protein enrichment small-capacity kit was purchased from Bio-Rad™ (Hercules, CA). Chromatography solvents were of LC-MS grade and purchased from Fisher Scientific (Waltham, MA). AAV reference materials, including AAV1 empty capsids, AAV5 empty capsids, AAV8 empty capsids, AAV9 empty capsids, AAV8 full capsids (74.8% full capsids) and AAV9 full capsids (82.3% full capsids) were purchased from Charles River Laboratory, expressed in HEK293 cells. Sodium deoxycholate (SDC), sodium lauroyl sarcosinate (SLS), 2-chloroacetamide (CAA), Tris(2-carboxyethyl)phosphine hydrochloride (TCEP) and a Proteomics Dynamic Range Standard Set (UPS2) were purchased from Sigma-Aldrich (St. Louis, MO). Iodoacetamide, dithiothreitol, trifluoracetic acid (TFA) and UltraPure 1 M Tris-HCl, pH 8.0, were purchased from Thermo Fisher. AAV 8 empty capsids with UPS2 spiked in were prepared by dilution of UPS2 (10.6 μg/ampule protein) in 32 μL water; subsequently, 2 μL diluted UPS2 was added to 500 μL AAV reference material with empty capsids. UPS2 is a dynamic range standard used in proteomics, comprising six mixtures of eight proteins spanning five orders of magnitude. This allows it to be utilized in assessing the enrichment factor of the recombinant proteins present in the UPS2 standard.

### Sample Preparation with Enrichment Beads

HCPs from AAV reference materials were enriched with ProteoMiner™ enrichment beads through a protocol similar to that described by Chen et al. (A Highly Sensitive LC-MS/MS Method for Targeted Quantitation of Lipase Host Cell Proteins in Biotherapeutics; J Pharm Sci 2021, 110, 3811-3818.) with modifications. The AAV reference materials were stored in PBS buffer containing 0.01% Pluronic F-68, pH 7. The viral particle titer of AAV reference materials with empty capsids ranged from 1.20×10¹² VP/mL to 2.30×10¹² VP/mL, and the viral genome titer of AAV8 reference material with full capsids was 7.97×10¹¹ GC/mL. The pH of AAV reference material empty capsids was adjusted by the addition of 50 mM acetic acid, pH 6, or 50 mM Tris-HCl, pH 8.0. Enrichment beads were washed three times with 200 μL wash buffer from the ProteoMiner™ enrichment kit and once with water, then resuspended in water. Subsequently, 10 μL of resuspended enrichment beads was added to AAV reference material and incubated with rotation at room temperature for 2.5 hours. The slurry of beads with enriched proteins was loaded onto an in-house made tip, centrifuged at 200×g for 5 minutes until dry, sequentially washed with 100 μL wash buffer three times and with water once, and eluted three times through the addition of 10 μL PTS buffer containing 12 mM SDC, 12 mM SLS, 10 mM TCEP and 40 mM CAA. The eluate was then denatured, reduced and alkylated in PTS buffer at 95° C. for 5 minutes, cooled to room temperature, diluted with 120 μL of 0.1 M Tris-HCl, pH 8.0, and digested with 10 ng trypsin overnight at 37° C. The digested peptides were acidified by addition of 10 μL of 10% TFA and centrifuged at 14,000×g for 20 minutes, and peptide-containing supernatant was collected for sequential desalting and nano LC-MS/MS analysis.

### Sample Preparation with Direct Digestion

Direct digestion was performed by addition of 10 μL of denaturing buffer containing 36 mM SDC/SLS, 30 mM TCEP and 120 mM CAA into 20 μL of AAV reference material and heating at 95° C. for 5 minutes. The denatured, reduced and alkylated AAV reference material was then diluted with 120 μL of 0.1 M Tris-HCl, pH 8.0, and digested with 100 ng trypsin overnight at 37° C. The digested peptides were acidified by the addition of 10 μL of 10% TFA and centrifuged at 14,000×g for 20 minutes. Peptide-containing supernatant was collected for sequential desalting and nano LC-MS/MS analysis.

### Nano LC-MS/MS analysis

The peptide mixture was resuspended in 12 μL 0.1% formic acid (FA) in water, and 10 μL was loaded onto an UltiMate 3000 RSLC nano LC system interfaced with an Orbitrap Exploris 480 mass spectrometer (Thermo Fisher Scientific). Peptides were sequentially loaded onto a trap column (20 cm×0.075 mm Acclaim PepMap 100 C18) at 5 μL/min, 40° C. and an analytical column (30 cm×0.075 mm, 1.7 μm, 100 Å, CoAnn Technologies) at 0.25 μL/min, 40° C. for desalting and separation. Mobile phase A was 0.1% FA in water, and mobile phase B was 0.1% FA in acetonitrile. The gradient started with 4% mobile phase B for 6 min, was increased to 20% B over 84 min and further increased to 36% B within 35 min, and then was steeply increased to 95% B in 9 min and held for 9 min. The Orbitrap Exploris was operated in data-dependent mode, with a full MS scan at 60,000 resolution at m/z 380-1600 and a predicted automatic gain control value of 3×10⁶ with a maximal injection time of 20 ms, and MS/MS event at a resolution of 15,000 resolutions, a predicted automatic gain control of 1×10⁵ and a maximal injection time of 60 ms at m/z 200-2000.

### Data Analysis

The raw MS data were searched against the UniProt Homo sapiens database (version 2022) with Proteome Discoverer software (version 2.4) by using the Sequest HT and Mascot search engines. The spectra were searched with 10 ppm mass tolerance for precursor ions and 0.02 Da for fragment ions, and tryptic digestion with a maximum of two missed cleavage sites. Carbamidomethylation of cysteines (+57.0214 Da) was included as a static modification, and oxidation (+15.9949 Da) on methionine and deamidation (+0.984 Da) on asparagine were included as variable modifications. The false discovery rates for proteins and peptides were set at 0.01, and HCPs were considered positively identified when at least two unique peptides were found.

### Results and Discussion

### Optimization of Bead Amount in the Enrichment Method

The effectiveness of enriching HCPs by using different bead amounts was evaluated with UPS2-spiked, pH adjusted AAV8 reference material with empty capsids. A 2.5 μL volume of 1 M acetic acid was added into 500 μL of AAV8 product to adjust the pH to ˜6 according to a ProteoMiner™ enrichment protocol. Subsequently, 1/16 UPS2 (0.66 μg protein) was spiked into 500 μL pH adjusted AAV8 empty capsids to evaluate the detection limit, and ½ of the ProteoMiner™ kit (10 μL beads), ⅕ of the ProteoMiner™ kit (4 μL beads), 1/20 of the ProteoMine™r kit (1 μL beads) or 1/50 of the ProteoMiner™ kit (0.4 μL beads) was incubated with 100 μL of pH adjusted AAV8 material with UPS2 spiked in. An average of 167 HCPs were detected after ProteoMiner™ enrichment, whereas only 45 HCPs were detected with direct digestion (FIG. 1A). All UPS2 standards above 6.25 fmol were detected after application of enrichment beads, whereas ¼ of these standards in the lower concentration (≤4 ng/ml) were not detectable through direct digestion (Table 1). No discernible variations were identified in the number of HCPs (FIG. 1A) or the number of unique peptides identified from UPS2 standards at 6.25 fmol (FIG. 1B) when different amounts of beads were applied. These results suggested that the enrichment efficiency, with respect to detection limit and sensitivity, was unaffected by the quantity of beads used. 1 μL of enrichment beads was used for all subsequent experiments.

### Evaluation of Sample Amount Used in the Experiment

Because of low titer during drug production, the quantity of AAV materials is often limited. Consequently, to evaluate HCP analysis and detection limit, we adjusted 20 μL, 50 μL and 100 μL of AAV8 and AAV9 reference materials to pH 6, then incubated them with 1 μL of ProteoMiner™ beads. The aim was to determine the minimum sample amount to evaluate the detection limit for the analysis of HCPs. The comparison between the number of detected HCPs and the detection limit was performed with AAV8 reference material with UPS2 spiked in and pH adjusted AAV9 reference material. The number of HCPs in the AAV8 and AAV9 reference material increased 60% when 50 μL or 100 μL of sample was used, and 46% when 20 μL was used. Unexpectedly, when 100 μL of sample was applied for enrichment, the number of HCPs detected remained the same as that when 50 μL was used (FIG. 1C). However, only five of eight UPS2 at 6.25 fmol were detected from the 50 μL sample, as compared with eight of eight UPS2 at 6.25 fmol detected from the 100 ML sample (FIG. 1D). A detection limit of 0.5 ng/ml was observed when 100 μL of sample was applied, and the detection limit increased as the amount of sample used for enrichment decreased. 100 μL of AAV reference material was used for all subsequent experimental procedures.

### Optimization of Binding Solution pH in the Enrichment Method

The pH of the binding solution for AAV products was evaluated. The AAV reference materials were originally stored in PBS buffer containing 0.01% Pluronic F-68, pH 7. The materials were adjusted to pH 6 by the addition of 50 mM acetic acid or to pH 8 by the addition of 50 mM Tris-HCl, pH 8.0. The enrichment was performed by incubation of 1 μL of ProteoMiner™ beads with 100 μL of AAV reference material. For AAV9 reference material with empty capsids, 191 HCPs were detected in the acidic condition, whereas 262 HCPs were detected in the neutral condition, and 109 HCPs were detected in the basic condition (FIG. 1E). To evaluate the neutral incubation condition for AAV serotypes for HCP enrichment, additional experiments on AAV1, AAV5 and AAV8 reference materials with empty capsids at pH 6 and pH 7 were performed. The number of HCPs detected from different AAV serotypes increased 15.6%-50.6% when AAV reference materials were incubated with enrichment beads under neutral conditions compared with acidic conditions (FIG. 1F). Therefore, neutral pH was chosen for HCP enrichment from AAV samples.

### Method Reproducibility

The reproducibility of the method was evaluated in triplicate under the experimental conditions, which involved incubation of 1 μL of ProteoMiner™ beads with 100 μL of AAV reference material in PBS buffer. Approximately 146 HCPs were identified with the enrichment method from the AAV8 reference material with full capsids, a number 4.3-fold higher than the number of HCPs identified through direct digestion. Across three runs, 131 common HCPs were identified, equivalent to 80.9% of all proteins (FIG. 2B). The high reproducibility suggested high confidence in protein identification-an attribute for HCP analysis methods.

### Application of Enrichment Beads Increases Identification of HCPs 5- to 10-Fold in All AAV Serotypes

The number of identified HCPs from AAV1, AAV5, AAV8, and AAV9 reference materials with empty capsids was 5 to 10 times higher with the enrichment method of the present study than with direct digestion (FIG. 3B). This enhancement was observed across all AAV serotypes. More than 80% of the newly identified HCPs were detected with the enrichment method, thus revealing less than 1.2% of missing proteins compared with the direct digestion approach. These findings indicated that the present method can comprehensively detect many HCPs present at low abundance, which are otherwise not detectable through direct digestion.

### Evaluation of Decreased Dynamic Range of UPS2-Spiked Standards in AAV9 Reference Material

To evaluate the dynamic range reduction of the enrichment method, 1/40 UPS2 (0.264 μg protein) was spiked into 200 μL AAV9 empty capsids. Seven UPS2 standards ranging from 0.1 ng/mL to 414.7 ng/mL were chosen for comparison. The concentration of each UPS2-spiked standard was calculated by comparison of the relative abundance of UPS2 standard peptides versus capsid protein peptides for samples prepared with the enrichment method and direct digestion method. Enrichment factors were calculated by comparison of the calculated concentration with the spiked-in concentration (Table 3). The enrichment factors of UPS2 standards prepared by direct digestion were close to 1, because no enrichment occurred. Some standards, such as serum albumin and peroxiredoxin, had enrichment factors slightly greater than 1, possibly due to the endogenous HCPs in the AAV reference material. The enrichment factor for ProteoMiner™-treated samples ranged from 1.5 to 25. For UPS2 above 1.6 ng/ml, the dynamic range between UPS2 peptide and AAV capsid protein peptide ranged between 3.5×10⁻⁴ and 3.0×10⁻¹ for the enrichment method and 2.5×10⁻⁴ and 3.0×10⁻² for direct digestion, thus demonstrating a 3.6-fold to 34.1-fold increase after enrichment. The signal changes in catalase peptide VFEHIGK²⁺ and ribosyldihydronicotinamide dehydrogenase [quinone] [Quinone oxidoreductase 2] [NQO2] peptide NVAVDELSR²⁺ are shown in FIG. 5A. For neddylin at 0.6 ng/ml and thioredoxin at 0.1 ng/mL, the dynamic range between UPS2 and AAV capsid protein peptides was 3.5×10⁻⁴ and 7.2×10⁻⁴ after enrichment. The dynamic range between these two UPS2 peptides and capsid protein peptides was predicted to be approximately 10⁻⁵ with direct digestion. Therefore, neddylin and thioredoxin, and probably other host cell proteins present at low ng/mL concentrations or below, were not detectable with direct digestion preparation. The MS2 spectrum of neddylin peptide EIEIDIEPTDKVER³⁺ at 0.6 ng/ml and thioredoxin peptide TAFQEALDAAGDK²⁺ at 0.1 ng/ml after enrichment (FIG. 5B), showed a substantial improvement in sensitivity in detecting these low-abundance HCPs (<1 ng/ml) with application of the enrichment method. The enrichment factors of the rest of spiked-in HCPs are reported in Table 3.

TABLE 3
Summary of the enrichment factor and the comparison of enrichment factor for spiked-in HCPs
in AAV reference material using ProteoMiner ™ enrichment method and direct digestion.

| | | | | Direct | | PM/ |
|---|---|---|---|---|---|---|
| | | | | digestion | PM | direct |
| | accession | | concentration | enrichment | enrichment | digestion |
| HCP | # | Mw | (ng/mL) | factor | factor | ratio |
| Carbonic anhydrase 1 | P00915 | 28738 | 179.6 | 1.03 | 3.73 | 3.61 |
| Carbonic anhydrase 2 | P00918 | 29115 | 182.0 | 4.23 | 24.70 | 5.84 |
| Complement C5 | P01031 | 8536 | 53.4 | 2.65 | 0.52 | 0.20 |
| [Complement C5a] | | | | | | |
| Hemoglobin alpha chain | P69905 | 15126 | 94.5 | 0.16 | 1.18 | 7.47 |
| Hemoglobin beta chain | P68871 | 15867 | 99.2 | 1.13 | 7.31 | 6.45 |
| Leptin | P41159 | 16158 | 101.0 | 0.42 | 2.40 | 5.76 |
| Serum Albumin | P02768 | 66357 | 414.7 | 1.35 | 13.53 | 10.00 |
| Ubiquitin | P62988 | 10597 | 66.2 | 3.37 | 4.28 | 1.27 |
| Catalase | P04040 | 59625 | 37.3 | 0.72 | 24.69 | 34.18 |
| Cytochrome b5 | P00167 | 16022 | 10.0 | 3.26 | 9.97 | 3.06 |
| Epidermal Growth Factor | P01133 | 6353 | 4.0 | — | 4.69 | — |
| Myoglobin C | P02144 | 17053 | 10.7 | 0.43 | 9.66 | 22.45 |
| NAD(P)H dehydrogenase | P15559 | 30736 | 19.2 | 2.57 | 4.81 | 1.87 |
| [quinone] 1 [DT Diaphorase] C | | | | | | |
| Peptidyl-prolyl cis-trans | P62937 | 20176 | 12.6 | 0.96 | 2.69 | 2.79 |
| isomerase A [Cyclophilin A] | | | | | | |
| Peroxiredoxin 1 | Q06830 | 21979 | 13.7 | 1.79 | 6.48 | 3.62 |
| Small ubiquitin-related | P63165 | 38815 | 24.3 | 1.15 | 0.51 | 0.44 |
| modifier 1 [SUMO-1] | | | | | | |
| Alpha-lactalbumin | P00709 | 14078 | 0.9 | — | 0.39 | — |
| Creatine kinase M-type | P06732 | 43101 | 2.7 | 3.28 | 4.52 | 1.38 |
| [CK-MM] | | | | | | |
| Histidyl-tRNA synthetase | P12081 | 58233 | 3.6 | — | 0.48 | — |
| [Jo-1] | | | | | | |
| Lysozyme C | P61626 | 14701 | 0.9 | 0.47 | 3.48 | 7.38 |
| Neddylin [Nedd8] | Q15843 | 9072 | 0.6 | — | 1.58 | — |
| Retinol-binding protein | P02753 | 21071 | 1.3 | — | 2.27 | — |
| Ribosyldihydronicotinamide | P16083 | 25821 | 1.6 | 1.16 | 6.40 | 5.51 |
| dehydrogenase [quinone] | | | | | | |
| [Quinone oxidoreductase 2] | | | | | | |
| [NQO2] | | | | | | |
| Ubiquitin-conjugating | P63279 | 18007 | 1.1 | — | 0.43 | — |
| enzyme E2 I [UbcH9] | | | | | | |
| Antithrombin-III | P01008 | 49039 | 0.3 | 2.30 | 6.76 | 2.95 |
| Thioredoxin | P10599 | 12429 | 0.1 | — | 25.57 | — |

### Complete Detergent Removal after Enrichment Bead Treatment

ProteoMiner™ technology was used to apply bead-based peptide ligands to enrich low-abundance proteins in drug products. Only proteins such as antibodies, AAV capsid proteins and host cell proteins bind the beads. Detergents, such as polysorbate or Pluronic F-68, do not bind the enrichment beads and thus were removed from the protein mixtures after enrichment. A molecular weight cut off filter cannot easily remove these detergents, and thus filtration did not decrease the interference from the detergent (FIG. 6). In addition, we observed a substantial loss of HCPs during buffer exchange, possibly because of their adhesion to the filter membrane. With direct digestion, the TIC chromatography and MS signals of Pluronic F-68 were more than 20 times higher than those of the viral protein peptides (FIGS. 7A-7B). This significant difference in signal levels posed a major challenge for the detection of HCP peptides, because the detectable HCP peptides had 100 to 10,000 times lower abundance than viral protein peptides. The TIC chromatography and corresponding MS spectrum of peptide mixtures after enrichment are displayed in (FIGS. 7C-7D). The detergent peaks were completely eliminated by the enrichment beads, and certain HCP peptides were observed at similar levels to viral protein peptides in the TIC profile.

### Conclusions

The inability to apply MS-based HCP analysis methods to gene therapy products in the field thus far is due to several challenges. These include limited sample quantity, interference from detergents used during viral vector production, and incomplete coverage of anti-HCP antibodies.

In this study, it was demonstrated that the disclosed methods can successfully detect HCPs in gene therapy viral vector products. Comprehensive optimization was performed to develop a sensitive HCP enrichment method for AAV products. The approach disclosed herein resulted in a 5- to 10-fold increase in the number of detected HCPs, and improved the dynamic range by as many as two orders of magnitude with respect to that of direct digestion. The method exhibited a reproducibility rate of 80.9%, and can detect HCPs as low as 0.1 ng/mL.

Through providing comprehensive and orthogonal information regarding AAV HCPs, this method may serve as a valuable tool for troubleshooting and identifying potentially problematic HCPs. Additionally, this method can aid in assessing the risks associated with AAV products.

In this research, a total of 847 Host Cell Proteins (HCPs) from Adeno-Associated Virus (AAV) types 1, 5, 8, and 9 were identified. This identification was achieved through the use of ProteoMiner™ enrichment in combination with nano LC-MS/MS. These HCPs can be categorized based on their biological functions. For instance, structural proteins such as desmoplakin, filamin-A, myosin-9, plectin, tublin, actin, vimentin, vinculin, and ezrin were identified. Transport proteins such as serum, Alpha-2-macroglobulin-like, Apolipoprotein, and haptoglobin were also found. Enzymes including Protein-glutamine, Caspase-14, Inosine-5′-monophosphate, Fructose-bisphosphate, Gamma-glutamylcyclotransferase, Cathepsin, Glyceraldehyde-3-phosphate, Arginase-1, Triosephosphate, Peroxiredoxin-2, Carboxypeptidase, and Transaldolase were identified as well. Binding proteins such as RNA-binding, annexin, leucine-rich, polypyrimidine and calcyclin-binding protein were also identified. Additionally, transcriptional proteins, heat shock proteins, immunoglobulins, glycoproteins, and ribonucleoproteins were identified among the HCPs (Table 4). HCPs including, but not limited to, peroxiredoxin 1, ubiquitin, actin and Hsc70, which may regulate cell functions, were also detected in AAV products (FIG. 4). The ProteoMiner™ enrichment method identified 584 previously unreported HCPs. The presence of HCP impurities can affect drug potency, and certain HCPs, such as cathepsin, can lead to viral protein degradation. Hence, the identification of these low-abundance HCPs, which might not be easily detected using other methods, may be used for troubleshooting, and optimizing cell line production and purification.

TABLE 4
Summary of gene name of HCPs identified from all serotypes of AAV reference materials prepared
with all different enrichment parameters and digestion condition (column 1). HCPs identified
from UPS2 standards using ProteoMiner ™ enrichment as negative control (column 2).
HCPs identified from AAV reference materials that does not contain UPS2 standards (column 3).
HCPs identified from all serotypes of AAV reference materials prepared with all different enrichment
parameters and digestion condition and had been reported in reference paper (column 4)

| | | HCPs identified from | |
|---|---|---|---|
| | | AAV reference material | HCPs detected from |
| All HCPs detected from | | without UPS2 spiked in | materials with/without |
| AAV reference materials | HCPs identified from | overlapped with HCPs | UPS2 standards spiked |
| with/without UPS2 | UPS2 standard after | identified from UPS2 | in after ProteoMiner ™ |
| standards spiked in | ProteoMiner ™ | standard after | enrichment and had |
| after ProteoMiner ™ | enrichment | ProteoMiner ™ enrichment | been reported in |
| enrichment | (negative control) | (positive control) | reference paper |
| 1433B | 1433S | 1433S | 1433B |
| 1433E | A0A087X0Q4 | A0A087X0Q4 | 1433E |
| 1433G | ACTS | ACTS | 1433G |
| 1433S | ALBU | ALBU | 1433S |
| 1433T | ANT3 | ANXA1 | 1433T |
| 1433Z | ANXA1 | ANXA2 | 1433Z |
| 1C02 | ANXA2 | ARGI1 | A2MG |
| 2A5A | ARGI1 | CASPE | AATC |
| 6PGD | CAH1 | CATA | ACBP |
| A0A087WVE2 | CAH2 | CYTA | ACPH |
| A0A087WWU8 | CASPE | DCD | ACTB |
| A0A087WZY1 | CATA | DESP | ACTN4 |
| A0A087X0Q4 | CO5 | DSC1 | AFAD |
| A0A0A0MRC0 | CYB5 | DSG1 | AGAL |
| A0A0A0MSI0 | CYTA | EF1A1 | ALDOA |
| A0A0A0MTS2 | DCD | FILA2 | AN32A |
| A0A0B4J231 | DESP | G3P | AN32B |
| A0A0B4J2B5 | DSC1 | H0YI49 | ANXA2 |
| A0A0D9SET8 | DSG1 | HBA | ANXA5 |
| A0A0G2JMB2 | EF1A1 | HORN | ANXA6 |
| A0A0G2JNZ0 | FILA2 | HSPB1 | APEX1 |
| A0A0U1RRM6 | G3P | HV118 | APLP2 |
| A0A1B0GTZ5 | GRP1 | HV335 | ARGI1 |
| A0A1B0GW82 | H0YI49 | IGHG1 | ASAH1 |
| A0A1C7CYX9 | HBA | IGHG4 | ASPG |
| A0A1W2PS58 | HBB | IGKC | ATPA |
| A0A286YEY1 | HBD | KV105 | ATPB |
| A1AG1 | HORN | LYSC | BASI |
| A1AT | HSPB1 | PLAK | BID |
| A20A4 | HV118 | PPIA | BLMH |
| A2MG | HV335 | PRDX1 | BOLA2 |
| A2ML1 | IGHG1 | RS27A | CALL3 |
| A4 | IGHG3 | S10A9 | CALM1 |
| A6XMV9 | IGHG4 | SBSN | CALR |
| AAAT | IGKC | SPB12 | CALU |
| AACT | KCRM | SPB3 | CASPE |
| AATC | KV105 | TGM3 | CATA |
| ACBP | LEP | VGFR2 | CATC |
| ACPH | LYSC | ZA2G | CAZA1 |
| ACSL1 | MYG | | CBX3 |
| ACTB | NEDD8 | | CBX5 |
| ACTBL | NPTX1 | | CDC37 |
| ACTN1 | NQO1 | | CH60 |
| ACTN4 | NQO2 | | CLCB |
| ACTS | PLAK | | CLH1 |
| AFAD | PPIA | | CLIC1 |
| AGAL | PRDX1 | | CNBP |
| AGRE3 | RB39B | | COF1 |
| AGRG4 | RETBP | | CPSF7 |
| AHNK | ROBO2 | | CREG1 |
| AHNK2 | RS27A | | CRKL |
| AL7A1 | S10A9 | | CYBP |
| AL9A1 | SBSN | | CYC |
| ALBU | SNTB1 | | CYTB |
| ALDH2 | SPB12 | | DCD |
| ALDOA | SPB3 | | DDB1 |
| AMOT | SUMO1 | | DDX17 |
| AMY2B | SYHC | | DHX36 |
| AN20B | TGM3 | | DLDH |
| AN32A | TSP1 | | DNS2A |
| AN32B | VGFR1 | | DPY30 |
| ANKR6 | VGFR2 | | E41L2 |
| ANX11 | ZA2G | | EF1G |
| ANXA1 | | | EF2 |
| ANXA2 | | | EIF3A |
| ANXA3 | | | EIF3G |
| ANXA4 | | | EIF3I |
| ANXA5 | | | ELAV1 |
| ANXA6 | | | ENOA |
| ANXA7 | | | ENPL |
| AP2B1 | | | EZRI |
| APEX1 | | | FABP5 |
| APLP2 | | | FAS |
| APMAP | | | FETUA |
| APOA1 | | | FILA |
| APOD | | | FKB10 |
| ARAP3 | | | FKBP4 |
| ARC1B | | | FLNA |
| ARGI1 | | | FUBP1 |
| ARP2 | | | FUS |
| ARP3 | | | G3P |
| ARPC2 | | | G6PI |
| ARPC3 | | | GDIB |
| ARPC4 | | | GGH |
| ARPC5 | | | GLU2B |
| ARSF | | | GNS |
| ASAH1 | | | GPT11 |
| ASPG | | | GRB2 |
| ASPM | | | GRN |
| AT12A | | | GRP75 |
| AT1A1 | | | GRP78 |
| AT2B1 | | | GSTP1 |
| ATM | | | H32 |
| ATPA | | | H90B2 |
| ATPB | | | HBA |
| ATPD | | | |
| ATX2L | | | HEBP2 |
| B1AK88 | | | HEXB |
| B2MG | | | HMGB1 |
| B3A2 | | | HMGB2 |
| B7Z6Z4 | | | HNRDL |
| B9A067 | | | HNRH1 |
| BAG2 | | | HNRH2 |
| BAG3 | | | HNRH3 |
| BASI | | | HNRPD |
| BASP1 | | | HNRPF |
| BID | | | HNRPK |
| BIN2 | | | HNRPL |
| BLMH | | | HNRPU |
| BOLA2 | | | HORN |
| BPI | | | HS71B |
| C9J2Y4 | | | HS90A |
| CA141 | | | HS90B |
| CADH1 | | | HSP72 |
| | | | HSP74 |
| | | | HSP7C |
| CAH6 | | | IF4A1 |
| CALL3 | | | IF4A2 |
| CALL5 | | | IF6 |
| CALM1 | | | IMB1 |
| CALR | | | IMDH2 |
| CALU | | | KCRB |
| CAMP | | | KCRU |
| CAN1 | | | KPYM |
| CAP1 | | | LA |
| CAP7 | | | LAMB1 |
| CAPG | | | LAMP1 |
| CAPZB | | | LAMP2 |
| CASPE | | | LASP1 |
| CATA | | | LDHA |
| CATB | | | LDHB |
| CATC | | | LG3BP |
| CATD | | | LRRF1 |
| CATG | | | LYSC |
| CATH | | | MA2B1 |
| CATL2 | | | MAOX |
| CATS | | | MARCS |
| CATZ | | | MDHC |
| CAZA1 | | | MDHM |
| CBPA4 | | | MPP10 |
| CBPM | | | MRP |
| CBPZ | | | MTPN |
| CBX3 | | | MYH9 |
| CBX5 | | | MYL6 |
| CD59 | | | MYL6B |
| CDC37 | | | NDKA |
| CDC42 | | | |
| CDD | | | NID1 |
| CDK19 | | | NPM |
| CDSN | | | NSF1C |
| CE350 | | | NTF2 |
| CEAM8 | | | NUDC |
| CELR1 | | | ODO2 |
| CERU | | | ODP2 |
| CH076 | | | ODPA |
| CH10 | | | ODPX |
| CH3L1 | | | PABP1 |
| CH60 | | | PCBP2 |
| CHD3 | | | PCNA |
| CHD4 | | | PCP |
| CHD9 | | | PDIA1 |
| CKAP4 | | | PDIA3 |
| CLCA | | | PDLI1 |
| CLCB | | | PEA15 |
| CLH1 | | | PGAM1 |
| CLIC1 | | | PIMT |
| CLUS | | | PIP |
| CNBP | | | PITH1 |
| CO3 | | | PLAK |
| CO4B | | | PLBL2 |
| | | | PLD3 |
| COEA1 | | | PNPH |
| COF1 | | | PPIA |
| COIA1 | | | PRC2C |
| COPB2 | | | PRDX1 |
| COR1A | | | PRDX3 |
| COR1C | | | PRDX4 |
| COX41 | | | PRDX6 |
| CPNE1 | | | PROF1 |
| CPNE3 | | | PRS6B |
| CPSF6 | | | PSA1 |
| CPSF7 | | | PSA2 |
| CRCT1 | | | PSA3 |
| CREG1 | | | PSA4 |
| CRERF | | | PSA5 |
| CRIS3 | | | PSA6 |
| CRKL | | | PSA7 |
| CRNN | | | PSB1 |
| CSTF2 | | | PSB2 |
| CY24A | | | PSB3 |
| | | | PSB4 |
| CYBP | | | PSB5 |
| CYC | | | PSB6 |
| CYFP1 | | | PSB7 |
| CYTA | | | PSMD4 |
| CYTB | | | PTBP1 |
| CYTM | | | PUR6 |
| CYTN | | | PZP |
| CYTS | | | RAB14 |
| D3DSM0 | | | RAB7A |
| DAZP1 | | | RBMX |
| DCD | | | RCN2 |
| DCP1B | | | RLA2 |
| DDB1 | | | RO60 |
| DDX17 | | | ROA1 |
| DEF1 | | | ROA2 |
| DEF3 | | | ROA3 |
| DESP | | | ROAA |
| DHB4 | | | RPAB3 |
| DHX36 | | | RSSA |
| DJB11 | | | S10A7 |
| DKK3 | | | S10A8 |
| DLDH | | | S10A9 |
| DMBT1 | | | S10AB |
| DMKN | | | SAHH |
| DMRTA | | | SAP |
| DNHD1 | | | SBSN |
| DNS2A | | | SCRB2 |
| DNSL2 | | | SET |
| DPP2 | | | SF01 |
| DPP3 | | | SF3B1 |
| DPY30 | | | SF3B2 |
| DR9C7 | | | SFPQ |
| DSC1 | | | SH3L3 |
| DSC3 | | | SMCE1 |
| DSG1 | | | SMD2 |
| DTD2 | | | SODC |
| DX39A | | | SPA12 |
| DYH5 | | | SPB12 |
| E41L2 | | | SRSF1 |
| E41L3 | | | SRSF3 |
| E7EMS2 | | | SRSF9 |
| E9PN91 | | | STIP1 |
| E9PRY8 | | | STMN1 |
| ECH1 | | | STMN2 |
| ECM1 | | | |
| ECP | | | SYEP |
| EEA1 | | | SYIC |
| EF1A1 | | | TAGL2 |
| EF1A2 | | | TB182 |
| EF1G | | | TBA1B |
| EF2 | | | TBB4B |
| EFHD2 | | | TBB5 |
| EGF | | | TCPB |
| EIF3A | | | TCPE |
| EIF3G | | | TCPQ |
| EIF3I | | | TERA |
| ELAF | | | TFG |
| ELAV1 | | | TGM3 |
| ELMO3 | | | THIO |
| ELNE | | | TITIN |
| EMAL4 | | | TKT |
| ENDD1 | | | TMOD3 |
| ENOA | | | TNR6B |
| ENPL | | | TPIS |
| EP15R | | | TPM4 |
| ERMP1 | | | TRFL |
| ERO1A | | | TXND5 |
| ERP29 | | | TYB4 |
| ESYT3 | | | UBP2L |
| EVPL | | | UCHL1 |
| EXOC5 | | | UCHL3 |
| EZRI | | | VIME |
| F2Z2A5 | | | VINC |
| F2Z393 | | | XP32 |
| F5H5D3 | | | ZA2G |
| F5H7Y6 | | | |
| F8VZG5 | | | |
| F8WCN3 | | | |
| FABP5 | | | |
| FAS | | | |
| FBX50 | | | |
| FETUA | | | |
| FGD6 | | | |
| FGR | | | |
| FHAD1 | | | |
| FIBA | | | |
| FIBB | | | |
| FIBG | | | |
| FILA | | | |
| FILA2 | | | |
| FINC | | | |
| FKB10 | | | |
| FKB1A | | | |
| FKBP4 | | | |
| FLNA | | | |
| FLNB | | | |
| FLOT1 | | | |
| FMNL1 | | | |
| FSCN1 | | | |
| FUBP1 | | | |
| FUBP2 | | | |
| FUS | | | |
| FYV1 | | | |
| G3P | | | |
| G3PT | | | |
| G3V251 | | | |
| G3V3E6 | | | |
| G6PD | | | |
| G6PI | | | |
| GCC2 | | | |
| GDIB | | | |
| GDIR2 | | | |
| GDPD3 | | | |
| GELS | | | |
| GGCT | | | |
| GGH | | | |
| GLCM | | | |
| GLRX1 | | | |
| GLU2B | | | |
| GMFG | | | |
| GNAI2 | | | |
| GNS | | | |
| GPNMB | | | |
| GPR98 | | | |
| GPT11 | | | |
| GRAN | | | |
| GRB2 | | | |
| GRN | | | |
| GRP75 | | | |
| GRP78 | | | |
| GRPE1 | | | |
| GSDMA | | | |
| GSHB | | | |
| GSTO1 | | | |
| GSTP1 | | | |
| GUAD | | | |
| H0Y3P2 | | | |
| H0Y4R1 | | | |
| H0YBL1 | | | |
| H0YH87 | | | |
| H0YIZ1 | | | |
| H0YMI6 | | | |
| H0YN42 | | | |
| H12 | | | |
| H15 | | | |
| H2A1C | | | |
| H2A1H | | | |
| H2A1J | | | |
| H2A3 | | | |
| H2AY | | | |
| H2B1A | | | |
| H2B1B | | | |
| H2B1D | | | |
| H2B1M | | | |
| H31 | | | |
| H31T | | | |
| H32 | | | |
| H3BTN5 | | | |
| H4 | | | |
| H7BYY1 | | | |
| H7BZJ3 | | | |
| H90B2 | | | |
| HBA | | | |
| HCFC1 | | | |
| HCLS1 | | | |
| HEBP2 | | | |
| HEM2 | | | |
| HESX1 | | | |
| HEXB | | | |
| HM13 | | | |
| HMGB1 | | | |
| HMGB2 | | | |
| HMGN2 | | | |
| HNRDL | | | |
| HNRH1 | | | |
| HNRH2 | | | |
| HNRH3 | | | |
| HNRNPAB | | | |
| HNRPD | | | |
| HNRPF | | | |
| HNRPK | | | |
| HNRPL | | | |
| HNRPU | | | |
| HORN | | | |
| HPT | | | |
| HS71B | | | |
| HS90A | | | |
| HS90B | | | |
| HSP72 | | | |
| HSP74 | | | |
| HSP7C | | | |
| HSPB1 | | | |
| HTRA2 | | | |
| HUTH | | | |
| HV118 | | | |
| HV169 | | | |
| HV335 | | | |
| HV353 | | | |
| HV69D | | | |
| HVC33 | | | |
| HXK3 | | | |
| I36RA | | | |
| IDE | | | |
| IDHC | | | |
| IDS | | | |
| IF4A1 | | | |
| IF4A2 | | | |
| IF6 | | | |
| IGHA1 | | | |
| IGHA2 | | | |
| IGHG1 | | | |
| IGHG2 | | | |
| IGHG4 | | | |
| IGHM | | | |
| IGJ | | | |
| IGKC | | | |
| IGLC1 | | | |
| IGLC3 | | | |
| IL1RA | | | |
| IL36G | | | |
| ILEU | | | |
| IMB1 | | | |
| IMDH1 | | | |
| IMDH2 | | | |
| IMPA1 | | | |
| IMPA2 | | | |
| INVO | | | |
| IQGA1 | | | |
| IRS4 | | | |
| ISK5 | | | |
| ITAM | | | |
| ITB1 | | | |
| ITPR2 | | | |
| J3KN67 | | | |
| J3KNL6 | | | |
| J3KRY1 | | | |
| J3QQX2 | | | |
| K7EJB9 | | | |
| KBTB8 | | | |
| KCC2D | | | |
| KCD12 | | | |
| KCRB | | | |
| KCRU | | | |
| KCTD5 | | | |
| KI2S1 | | | |
| KLC3 | | | |
| KLK10 | | | |
| KLK5 | | | |
| KLK7 | | | |
| KLK8 | | | |
| KNTC1 | | | |
| KPYM | | | |
| KV105 | | | |
| KV320 | | | |
| KVD12 | | | |
| KVD28 | | | |
| KVD39 | | | |
| LA | | | |
| LACRT | | | |
| LAMB1 | | | |
| LAMP1 | | | |
| LAMP2 | | | |
| LAP2B | | | |
| LASP1 | | | |
| LAT1 | | | |
| LCE2C | | | |
| LCN1 | | | |
| LDHA | | | |
| LDHB | | | |
| LEG3 | | | |
| LEG7 | | | |
| LG3BP | | | |
| LIN7A | | | |
| LKHA4 | | | |
| LMNA | | | |
| LMNB1 | | | |
| LMNB2 | | | |
| LORI | | | |
| LOXE3 | | | |
| LOXH1 | | | |
| LRRF1 | | | |
| LSP1 | | | |
| LX12B | | | |
| LXN | | | |
| LY6D | | | |
| LYAG | | | |
| LYPD3 | | | |
| LYPD5 | | | |
| LYSC | | | |
| MA2B1 | | | |
| MAOX | | | |
| MARCS | | | |
| MDHC | | | |
| MDHM | | | |
| MGST2 | | | |
| ML12A | | | |
| MMP8 | | | |
| MMP9 | | | |
| MNDA | | | |
| MOES | | | |
| MORC2 | | | |
| MOT1 | | | |
| MPCP | | | |
| MPDZ | | | |
| MPP10 | | | |
| MRCKG | | | |
| MRP | | | |
| MSLN | | | |
| MTAP | | | |
| MTPN | | | |
| MUCL1 | | | |
| MXRA5 | | | |
| MYH14 | | | |
| MYH9 | | | |
| MYL6 | | | |
| MYL6B | | | |
| NADC | | | |
| NADK | | | |
| NAMPT | | | |
| NCF1C | | | |
| NCF2 | | | |
| NCL | | | |
| NCOA2 | | | |
| NDKA | | | |
| NDKB | | | |
| NEK4 | | | |
| NENF | | | |
| NEUR2 | | | |
| NFIB | | | |
| NGAL | | | |
| NID1 | | | |
| NKPD1 | | | |
| NONO | | | |
| NPC1 | | | |
| NPM | | | |
| NSF1C | | | |
| NSMA2 | | | |
| NTF2 | | | |
| NU153 | | | |
| NU214 | | | |
| NUCB1 | | | |
| NUCB2 | | | |
| NUDC | | | |
| NUDT5 | | | |
| NUFP2 | | | |
| NUMA1 | | | |
| NUP50 | | | |
| NUP98 | | | |
| OBSCN | | | |
| ODO2 | | | |
| ODP2 | | | |
| ODPA | | | |
| ODPX | | | |
| OTU1 | | | |
| P06748 | | | |
| PA24E | | | |
| PABP1 | | | |
| PADI1 | | | |
| PADI3 | | | |
| PADI4 | | | |
| PAI2 | | | |
| PAR1 | | | |
| PARP1 | | | |
| PAX3 | | | |
| PB1 | | | |
| PCBP1 | | | |
| PCBP2 | | | |
| PCNA | | | |
| PCP | | | |
| PDIA1 | | | |
| PDIA3 | | | |
| PDIA4 | | | |
| PDIP2 | | | |
| PDLI1 | | | |
| PDX1 | | | |
| PEA15 | | | |
| PEBP1 | | | |
| PEPL | | | |
| PERM | | | |
| PFD2 | | | |
| PFD3 | | | |
| PGAM1 | | | |
| PGAM2 | | | |
| PGBM | | | |
| PGK1 | | | |
| PGM1 | | | |
| PGRP1 | | | |
| PGS2 | | | |
| PHB2 | | | |
| PHLD | | | |
| PIGR | | | |
| PIMT | | | |
| PIP | | | |
| PITH1 | | | |
| PKP1 | | | |
| PLAK | | | |
| PLB1 | | | |
| PLBL1 | | | |
| PLBL2 | | | |
| PLCB2 | | | |
| PLCB4 | | | |
| PLCX1 | | | |
| PLD3 | | | |
| PLEC | | | |
| PLP2 | | | |
| PLSL | | | |
| PNPH | | | |
| POF1B | | | |
| PPAP | | | |
| PPGB | | | |
| PPIA | | | |
| PPIL1 | | | |
| PPIL3 | | | |
| PPT1 | | | |
| PR40A | | | |
| PRC2C | | | |
| PRDX1 | | | |
| PRDX2 | | | |
| PRDX3 | | | |
| PRDX4 | | | |
| PRDX5 | | | |
| PRDX6 | | | |
| PREX1 | | | |
| PROF1 | | | |
| PROL1 | | | |
| PROL4 | | | |
| PRS6B | | | |
| PRS7 | | | |
| PSA | | | |
| PSA1 | | | |
| PSA2 | | | |
| PSA3 | | | |
| PSA4 | | | |
| PSA5 | | | |
| PSA6 | | | |
| PSA7 | | | |
| PSB1 | | | |
| PSB2 | | | |
| PSB3 | | | |
| PSB4 | | | |
| PSB5 | | | |
| PSB6 | | | |
| PSB7 | | | |
| PSB9 | | | |
| PSMD4 | | | |
| PSMD9 | | | |
| PSPC1 | | | |
| PTBP1 | | | |
| PTN11 | | | |
| PTN6 | | | |
| PUR6 | | | |
| PYGB | | | |
| PYGL | | | |
| PYGO2 | | | |
| PZP | | | |
| Q01105 | | | |
| Q5VVL7 | | | |
| QSOX1 | | | |
| RAB10 | | | |
| RAB14 | | | |
| RAB1B | | | |
| RAB5B | | | |
| RAB7A | | | |
| RABP2 | | | |
| RAC2 | | | |
| RAN | | | |
| RAP1A | | | |
| RB11A | | | |
| RBMX | | | |
| RCN2 | | | |
| RET4 | | | |
| RETN | | | |
| RFOX2 | | | |
| RHG01 | | | |
| RISC | | | |
| RL10A | | | |
| RLA2 | | | |
| RN167 | | | |
| RNAS7 | | | |
| RO60 | | | |
| ROA1 | | | |
| ROA2 | | | |
| ROA3 | | | |
| ROAA | | | |
| ROBO3 | | | |
| RPAB3 | | | |
| RPIA | | | |
| RRP12 | | | |
| RS18 | | | |
| RS2 | | | |
| RS21 | | | |
| RS27A | | | |
| RS3 | | | |
| RSSA | | | |
| RUVB1 | | | |
| RXFP1 | | | |
| RYR3 | | | |
| S100P | | | |
| S10A6 | | | |
| S10A7 | | | |
| S10A8 | | | |
| S10A9 | | | |
| S10AB | | | |
| S10AE | | | |
| S10AG | | | |
| S4A8 | | | |
| S4R3E9 | | | |
| S61A1 | | | |
| SAHH | | | |
| SAP | | | |
| SAP3 | | | |
| SAPL1 | | | |
| SBP1 | | | |
| SBSN | | | |
| SCAPE | | | |
| SCEL | | | |
| SCRB2 | | | |
| SCUB2 | | | |
| SDCB1 | | | |
| SDF2L | | | |
| SEM5A | | | |
| SEMG1 | | | |
| SESQ1 | | | |
| SET | | | |
| SF01 | | | |
| SF3A1 | | | |
| SF3B1 | | | |
| SF3B2 | | | |
| SF3B4 | | | |
| SFPQ | | | |
| SG1D1 | | | |
| SG2A1 | | | |
| SG2A2 | | | |
| SH3L3 | | | |
| SIAE | | | |
| SLUR1 | | | |
| SLUR2 | | | |
| SMCE1 | | | |
| SMD2 | | | |
| SMRC2 | | | |
| SNG2 | | | |
| SNP23 | | | |
| SNRPD2 | | | |
| SNRPD3 | | | |
| SODC | | | |
| SPA12 | | | |
| SPB12 | | | |
| SPB13 | | | |
| SPB3 | | | |
| SPB4 | | | |
| SPB6 | | | |
| SPB7 | | | |
| SPHM | | | |
| SPR1A | | | |
| SPR1B | | | |
| SPR2D | | | |
| SPR2E | | | |
| SPRR4 | | | |
| SPTN2 | | | |
| SPTN4 | | | |
| SRSF1 | | | |
| SRSF3 | | | |
| SRSF7 | | | |
| SRSF9 | | | |
| SSXT | | | |
| STIP1 | | | |
| STMN1 | | | |
| STMN2 | | | |
| STOM | | | |
| STS | | | |
| STX4 | | | |
| STXB4 | | | |
| SUV3 | | | |
| SYEP | | | |
| SYIC | | | |
| SYPL1 | | | |
| TAGL2 | | | |
| TALDO | | | |
| TB182 | | | |
| TBA1A | | | |
| TBA1B | | | |
| TBB4B | | | |
| TBB5 | | | |
| TBCA | | | |
| TCPB | | | |
| TCPD | | | |
| TCPE | | | |
| TCPQ | | | |
| TCPZ | | | |
| TERA | | | |
| TFG | | | |
| TGM1 | | | |
| TGM3 | | | |
| TGM5 | | | |
| THIO | | | |
| TIM13 | | | |
| TIM8A | | | |
| TITIN | | | |
| TKT | | | |
| TLE3 | | | |
| TLN1 | | | |
| TMEDA | | | |
| TMOD3 | | | |
| TNR6B | | | |
| TO6BL | | | |
| TOLIP | | | |
| TPIS | | | |
| TPM4 | | | |
| TPP1 | | | |
| TRFE | | | |
| TRFL | | | |
| TRI29 | | | |
| TRNK1 | | | |
| TRY2 | | | |
| TUFT1 | | | |
| TXLNA | | | |
| TXND5 | | | |
| TYB4 | | | |
| TYPH | | | |
| UB2V1 | | | |
| UBA1 | | | |
| UBE2N | | | |
| UBIQ | | | |
| UBP2L | | | |
| UBQL2 | | | |
| UCHL1 | | | |
| UCHL3 | | | |
| UN93B | | | |
| UNK | | | |
| USO1 | | | |
| VAMP2 | | | |
| VASP | | | |
| VAT1 | | | |
| VATA | | | |
| VATB2 | | | |
| VDAC1 | | | |
| VDAC2 | | | |
| VGFR1 | | | |
| VIME | | | |
| VINC | | | |
| VNN2 | | | |
| VP13A | | | |
| WBP2 | | | |
| WDR1 | | | |
| WDR62 | | | |
| WFD12 | | | |
| WFDC5 | | | |
| WNK1 | | | |
| WTIP | | | |
| XP32 | | | |
| YAP1 | | | |
| YLPM1 | | | |
| ZA2G | | | |
| ZC3HE | | | |
| ZC4H2 | | | |
| ZG16B | | | |
| ZMYM6 | | | |
| ZN710 | | | |
| ZYX | | | |

Those skilled in the art will appreciate that numerous changes and modifications can be made to the preferred embodiments disclosed herein and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

What is claimed is:

1. A method of enriching, identifying and/or characterizing at least one host cell protein (HCP) impurity in a sample containing at least one viral vector, comprising:
(a) contacting a sample including at least one HCP impurity to a solid support, wherein said solid support has attached thereto a library of peptide ligands capable of interacting with said at least one HCP impurity generating a slurry;
(b) washing said slurry containing at least one HCP impurity to remove unbound material;
(c) eluting the bound HCPs to produce an enriched HCP sample;
(d) subjecting said enriched HCPs to enzymatic digestion conditions to produce a peptide digest; and
(e) subjecting said peptide digest to mass spectrometry/mass spectrometry (MS/MS) analysis to enrich, identify and/or characterize said at least one HCP impurity.

2. The method of claim 1, wherein said enriched HCPs are subjected to a denaturant to produce a denatured sample and wherein said denaturant comprises heat, high pH, low pH, reducing agents, or chaotropic agents.

3. The method of claim 1, wherein said enriched HCPs are subjected to an alkylating agent to produce an alkylated sample.

4. The method of claim 3, wherein said alkylating agent comprises iodoacetamide (IOA/IAA), chloroacetamide (CAA), acrylamide (AA), N-ethylmaleimide (NEM), methyl methanethiosulfonate (MMTS), 4-vinylpyridine, or combinations thereof.

5. The method of claim 1, wherein said enriched HCPs are subjected to a reducing agent to produce a reduced sample and wherein said reducing agent comprises dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof.

6. The method of claim 1, wherein said enriched HCPs are subjected to a denaturant, an alkylating agent, and a reducing agent to produce a denatured, reduced, and alkylated sample.

7. The method of claim 1, wherein the viral vector is an AAV vector comprising a serotype selected from AAV1, AAV2, AAV3, AAV4, AAV5, AAV6, AAV7, AAV8, AAVrh8, AAV2/8, AAV9, AAV10, AAV11, AAV12, variations thereof and combinations thereof.

8. The method of claim 1, wherein the amount of sample containing at least one host cell protein impurity is about 20 μL to about 100 μL.

9. The method of claim 1, wherein said enzymatic digestion conditions comprise contacting said denatured, reduced and alkylated sample with at least one digestive enzyme.

10. The method of claim 9, wherein said at least one digestive enzyme comprises trypsin.

11. The method of claim 1, wherein said mass spectrometer is an electrospray ionization mass spectrometer, nano-electrospray ionization mass spectrometer, or a triple quadrupole mass spectrometer.

12. The method of claim 1, wherein said mass spectrometer is coupled to a liquid chromatography system.

13. A method of enriching, identifying and/or characterizing at least one host cell protein (HCP) impurity in a sample containing at least one viral vector, comprising:
(a) contacting a sample including at least one HCP impurity to a solid support, wherein said solid support has attached thereto a library of peptide ligands capable of interacting with said at least one HCP impurity generating a slurry;
(b) washing said slurry containing at least one HCP impurity to remove unbound material;
(c) eluting bound HCPs to produce an enriched HCP sample;
(d) subjecting said enriched HCP sample to a denaturant to produce a denatured sample;
(e) subjecting said denatured sample to a reducing agent to produce a denatured and reduced sample;
(f) subjecting said denatured and reduced sample to an alkylating agent to produce a denatured, reduced and alkylated sample;
(g) subjecting said denatured, reduced and alkylated sample to enzymatic digestion conditions to produce a peptide digest; and
(h) subjecting said peptide digest to liquid chromatography-mass spectrometry/mass spectrometry (LC-MS/MS) analysis to identify and/or characterize said at least one HCP impurity.

14. The method of claim 13, wherein the viral vector is an AAV vector comprising a serotype selected from AAV1, AAV2, AAV3, AAV4, AAV5, AAV6, AAV7, AAV8, AAVrh8, AAV2/8, AAV9, AAV10, AAV11, AAV12, variations thereof and combinations thereof.

15. The method of claim 14, wherein the amount of sample containing at least one host cell protein impurity is about 20 μL to about 100 μL.

16. The method of claim 13, wherein said denaturant comprises heat, high pH, low pH, reducing agents, or chaotropic agents.

17. The method of claim 13, wherein said alkylating agent comprises iodoacetamide (IOA/IAA), chloroacetamide (CAA), acrylamide (AA), N-ethylmaleimide (NEM), methyl methanethiosulfonate (MMTS), 4-vinylpyridine, or combinations thereof.

18. The method of claim 13, wherein said reducing agent comprises dithiothreitol (DTT), β-mercaptoethanol, Ellman's reagent, hydroxylamine hydrochloride, sodium cyanoborohydride, tris(2-carboxyethyl)phosphine hydrochloride (TCEP-HCl), or combinations thereof.

19. The method of claim 13, wherein said enzymatic digestion conditions comprise contacting said denatured, reduced, and alkylated sample to at least one digestive enzyme, optionally wherein said at least one digestive enzyme comprises trypsin.

20. The method of claim 13, wherein said mass spectrometry system is electrospray ionization mass spectrometer, nano-electrospray ionization mass spectrometer, or an Orbitrap-based mass spectrometer and wherein said mass spectrometer is coupled to said liquid chromatography system.
